(19) United States
(12) Patent Application Publication
(10) Pub. No.: US 2021/0228114 A1
(43) Pub. Date: Jul. 29, 2021

(54) ADJUNCT DATA TO IMPROVE THE PERFORMANCE OF A CONTINUOUS GLUCOSE MONITORING SYSTEM

(71) Applicant: WaveForm Technologies, Inc., Salem, NH (US)

(72) Inventors: Mihailo V. REBEC, Portland, OR (US); Hua CAI, Salem, NH (US); Robert BRUCE, Beaverton, OR (US); Ralph DUTT-BALLERSTADT, Portland, OR (US)

(73) Assignee: WaveForm Technologies, Inc., Salem, NH (US)

(21) Appl. No.: 17/156,372

(22) Filed: Jan. 22, 2021

Related U.S. Application Data
(60) Provisional application No. 62/964,975, filed on Jan. 23, 2020.

(51) Int. Cl. A61B 5/145; A61B 5/00; A61B 5/024

(52) U.S. Cl. CPC A61B 5/14532; A61B 5/746; A61B 5/4839; A61B 5/1451; A61B 2562/0219; A61B 5/7267; A61B 2562/0247; A61B 2562/0271; A61B 5/024

(57) ABSTRACT

Systems and methods for operating continuous analyte monitoring (CAM) device are provided. In one example, a method comprises converting a first analyte data stream into analyte values reflective of a biological concentration of the analyte, obtaining one or more additional data streams from one or more adjunctive sensors, inferring based on the first data stream and the one or more additional data streams that conversion of the first data stream into analyte values is predicted to be inaccurate, and taking mitigating action to avoid inaccurate analyte values from being reported to a user. In this way, corrective measures can be taken to improve overall CAM device operation, quality of data provided via a CAM device may be enhanced, and user health and safety profile associated with the continuous analyte monitoring device may be improved.

### CROSS-REFERENCE TO RELATED APPLICATION

This application claims the priority benefit of the earlier filing data of U.S. Provisional Application No. 62/964,975, filed Jan. 23, 2020, which is hereby incorporated herein by reference in its entirety.

### TECHNICAL FIELD

Embodiments herein relate to the field of continuous analyte monitoring, and more specifically, to controlling operational aspects of a continuous analyte monitoring system, including estimates blood glucose concentration, based at least in part on adjunctive data.

### BACKGROUND

Blood glucose levels are mainly regulated by a hormone called insulin, secreted from the pancreatic β-cells. In type 1 diabetes, insulin secretion is lost due to an autoimmune process that destroys the β-cells. Type 1 diabetes is treated with life-long insulin replacement therapy that aims at maintaining glucose levels in a tight target range in order to avoid long-term macro- and micro-vascular complications. However, delivering the right amount of insulin is challenging, in part due to the intermittent nature of traditional glucose meters (4-7 measurements per day). Glucose levels often experience large swings in short periods of time, resulting in many unrecognized hypoglycemia (low glucose levels) and hyperglycemia (high glucose levels). Similar issues are prevalent in patients with Type 2 diabetes, in which the body either resists the effects of insulin, or doesn't produce enough insulin to maintain normal glucose levels.

Continuous glucose monitoring systems were developed to regularly measure glucose levels (e.g., every 5 minutes), and thus overcome the shortcomings of traditional blood glucose meters. Continuous glucose monitoring systems can improve glucose control, and when combined with insulin pumps, form artificial pancreas systems that have the potential to revolutionize diabetes care. Yet reliance on continuous glucose monitoring systems inherently requires that glucose values determined via such systems accurately reflect glucose values actually present in the blood of subjects using such systems. Hence, there is a need to identify particular physiologic and/or environmental conditions which can adversely impact accuracy of continuous glucose monitoring systems, and to provide solutions for correcting or otherwise compensating for such conditions so as to improve overall operation of continuous glucose monitoring systems.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is a schematic representation of an analyte sensor system in accordance with various embodiments;

FIG. 2 is a schematic representation of a networked continuous analyte monitoring (CAM) system for the implementation of the methods disclosed herein;

FIG. 3 illustrates a high-level example method for controlling operation of a continuous analyte monitoring system in accordance with various embodiments;

FIG. 4 illustrates a high-level example process flow for estimating analyte concentration based on one or more of an analyte sensor, one or more adjunct sensor(s), and/or other relevant historical data;

FIG. 5 illustrates a physical location of an analyte sensor and its proximity to one or more adjunct sensor(s) on the body of a user;

FIG. 6 depicts an example timeline illustrating control of an actuator associated with a continuous glucose monitoring (CGM) system based on data obtained from one or more adjunct sensors;

FIG. 7 illustrates a high-level example method for improving data quality of a continuous analyte monitoring system, in accordance with various embodiments;

FIG. 8 depicts an example timeline illustrating control of an actuator associated with a CGM system based on data obtained from an accelerometer positioned within a predetermined distance from a continuous glucose sensor; and

FIGS. 9A-9B are graphs showing a combination of data obtained from an analyte sensor, temperature sensor, and accelerometer over a first 24 hour time period (FIG. 9A) and a second 24 hour time period (FIG. 9B).

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense.

Various operations may be described as multiple discrete operations in turn, in a manner that may be helpful in understanding embodiments; however, the order of description should not be construed to imply that these operations are order-dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

### I. Overview of Several Embodiments

In one aspect, a method comprises obtaining a first data stream corresponding to a concentration of an analyte in a biological fluid from an analyte sensor; converting the first data stream into analyte values reflective of the concentration of the analyte; obtaining one or more additional data streams from one or more adjunctive sensors; inferring, based on the first data stream and the one or more additional data streams, that conversion of the first data stream into analyte values is predicted to be inaccurate; and taking mitigating action to avoid inaccurate analyte values from being reported to a user. The one or more adjunctive sensors may be selected from a pressure sensor, a temperature sensor, an accelerometer, and a heart rate sensor.

In an embodiment of the method, inferring that conversion of the first data stream into analyte values is predicted to be inaccurate further comprises comparing the first data stream and the one or more additional data streams to a set of historical data. The set of historical data may be computationally processed to reveal patterns of data corresponding to analyte and adjunctive sensor data streams indicative of circumstances where conversion of acquired data into analyte values is inaccurate. In one example, computationally processing the set of historical data may further comprise performing computational operations selected from one or more of supervised learning, unsupervised learning, and reinforcement learning, on the set of historical data.

In an embodiment of the method, taking mitigating action may further comprise applying a correction factor to a function that converts the first data stream into analyte values, and reporting corrected analyte values to the user. In an example, reporting corrected analyte values to the user may further comprise providing to the user an indication of a confidence level of the corrected analyte values.

In an embodiment of the method, the method may further comprise preventing an alarm associated with the analyte sensor from being activated when the corrected analyte values do not exceed one or more predetermined analyte value thresholds.

In an embodiment of the method, taking mitigating action may further comprise alerting the user that the analyte values are currently inaccurate, and providing a request to the user to obtain analyte values via another method that does not involve the analyte sensor.

In an embodiment of the method, the analyte sensor is a continuous analyte sensor implanted interstitially in skin of the user. In one example, the analyte may be glucose. In such an example, the continuous analyte sensor may be comprised of a membrane system defined herein as a permeable or semi-permeable membrane that can be comprised of two or more domains typically constructed of materials of a few microns thickness or more (or less in some examples). At least a portion of the membrane may be permeable to oxygen, and optionally permeable to glucose. In one example, the membrane system comprises an immobilized glucose oxidase enzyme, that enables an electrochemical reaction to occur to measure a concentration of glucose.

In another aspect, disclosed is a method of controlling an actuator associated with a continuous glucose sensor system. The method may comprise 1) predicting that conversion of a raw data stream obtained from a continuous glucose sensor interstitially implanted into skin of a user is expected to result in reporting of inaccurate glucose values that are not representative of an actual concentration of glucose sensed by the continuous glucose sensor; 2) applying a correction factor to a function that converts the raw data stream into glucose values, to obtain corrected glucose values that more accurately reflect the actual concentration of glucose sensed by the continuous glucose sensor, within a predetermined margin of error; 3) controlling the actuator in a first mode when the corrected glucose values do not exceed one or more predetermined glucose value thresholds; and 4) controlling the actuator in a second mode when the corrected glucose values exceed at least one of the predetermined glucose value thresholds.

In an embodiment of the method, the actuator may be an alarm that is audible and/or vibrational. Controlling the alarm in the first mode may include preventing the alarm from being activated. Controlling the alarm in the second mode may include activating the alarm to alert the user of a hypoglycemic or hyperglycemic event.

In another embodiment of the method, the actuator may be an insulin pump operably coupled to the continuous glucose sensor system, and capable of delivering a variable amount of insulin to the user as a function of determined glucose values. In such an example, controlling the insulin pump in the first mode may include maintaining the insulin pump off. Controlling the insulin pump in the second mode may include activating the insulin pump as a function of an extent to which the corrected glucose values exceeds one of the predetermined glucose value thresholds corresponding to a hyperglycemic event.

In an embodiment of the method, the predicting is based at least in part on data 1) currently being acquired from the continuous glucose sensor and from at least one adjunct sensor; and 2) a correlation of the data currently being acquired from both the continuous glucose sensor and the at least one adjunct sensor with previously obtained data that includes data obtained from the at least one adjunct sensor and the continuous glucose sensor or other similar adjunct sensor(s) and continuous glucose sensor(s) used in previous sensor sessions. In one such example, the one or more adjunct sensors may include a pressure sensor, a temperature sensor, and an accelerometer. In an example, each of the one or more adjunct sensors and the continuous glucose sensor are all positioned on the user within a same area defined by a radius R. In examples, radius R may be 2 cm or less. In some examples, the method may further comprise processing the previously obtained data via a computational strategy capable of learning when particular continuous glucose sensor data trends in combination with particular adjunct sensor data trends lead to inaccurate glucose values in absence of the corrective factor.

In an embodiment of the method, the method further comprises providing a confidence level reflective of the corrected glucose values. In some examples, the method includes adjusting the one or more predetermined glucose value thresholds as a function of the confidence level of the corrected glucose values. For example, the one or more thresholds may be adjusted to a greater extent when confidence levels are lesser, and may be adjusted to lesser extents when confidence levels are higher. In examples, the adjusting of the one or more thresholds comprises adjusting the one or more thresholds to more conservative thresholds (e.g., lowering a threshold at which an alarm may be triggered based on a determined concentration of analyte).

In another aspect, disclosed herein is a glucose sensor system. The glucose sensor system may comprise a continuous glucose sensor for interstitial implantation into skin of a user; one or more adjunct sensors selected from a pressure sensor, a temperature sensor, an accelerometer, and a heart rate sensor; and one or more actuatable components. The system may further comprise a computing device storing instructions in non-transitory memory that, when executed, cause the computing device to retrieve a first data stream from the continuous glucose sensor; retrieve one or more additional data streams from the one or more adjunct sensors; compare the first data stream and the one or more additional data streams to a historical data set comprising learned associative patterns of data corresponding to previously acquired data from the continuous glucose sensor and the one or more adjunct sensors, wherein the learned associative patterns are related to instances where conversion of the first data stream into glucose values results in glucose values that are not reflective of actual glucose concentrations measured via the continuous glucose sensor; predict, based on the comparing, that converting the first data stream into glucose values is expected to result in glucose values that are not reflective of the actual glucose concentrations measured via the continuous glucose sensor; initiate a compensation operation to yield corrected glucose values that are reflective of the actual glucose concentrations within some margin of error; and control at least one of the one or more actuatable components based on the corrected glucose values under circumstances where the compensation operation can yield the corrected glucose values that are reflective of the actual glucose concentrations within the margin of error.

In an embodiment, the system may further comprise a display operably linked to the computing device. In such an example, the computing device may store further instructions to send the corrected glucose values to the display device for viewing by the user, along with an indication that the values correspond to the corrected glucose values. In an example, the indication that the values correspond to the corrected glucose values includes one or more of displaying the corrected glucose values in a flashing manner as opposed to a stable manner; displaying the corrected glucose values in a color that is different from when non-corrected glucose values are displayed; and displaying a message along with the corrected glucose values that provides the user with information indicating that values displayed correspond to the corrected glucose values.

In an embodiment of the system, the computing device stores further instructions to prevent a calibration operation from being initiated during a time frame when the first data stream is being converted via the compensation operation to the corrected glucose values. The computing device may store further instructions to reschedule the calibration operation for another time under conditions where the calibration operation was scheduled to occur during the time frame when the first data stream is being converted to the corrected glucose values.

In an embodiment of the system, the computing device stores further instructions to assign a confidence level to the corrected glucose values, and control at least one of the one or more actuatable components based in part on the confidence level assigned to the corrected glucose values.

In an embodiment of the system, the actuatable component may be an audible and/or vibrational alarm configured to alert the user of a biological event related to blood glucose levels. In such an example, the computing device may store further instructions to prevent the alarm from being activated provided that the corrected glucose values do not exceed one or more predetermined glucose value thresholds, and activate the alarm in response to the corrected glucose values exceeding the one or more predetermined glucose value thresholds for a predetermined amount of time.

In an embodiment of the system, the actuatable component may be an insulin pump operably linked to the computing device. In such an example, the computing device may store further instructions to prevent the insulin pump from being activated provided that the corrected glucose values do not exceed a hyperglycemic threshold, and activate the insulin pump according to stored instructions in response to the corrected glucose values exceeding the hyperglycemic threshold for a predetermined amount of time.

In another embodiment of the system, the computing device stores further instructions to compare the first data stream and the one or more additional data streams to the historical data set, where the historical data set additionally comprises learned associative patterns of data. The learned associative patterns of data may be related to instances where conversion of the first data stream into glucose values results in glucose values that are accurately reflective of actual glucose concentrations measured via the continuous glucose sensor. In such an example, the system may control at least one of the one or more actuatable components based on non-corrected glucose values under circumstances where it is predicted that the non-corrected glucose values are reflective of actual glucose concentrations.

In another aspect, a method for a continuous analyte sensor system comprises determining, based on a first data stream retrieved from a continuous analyte sensor and at least a second data stream retrieved from an adjunct sensor, that a user of the continuous analyte sensor system has adopted a posture that results in the first data stream inaccurately reflecting a concentration of an analyte sensed by the continuous analyte sensor; providing, based on at least the first data stream and the second data stream, compensated analyte values that accurately reflect the concentration of the analyte sensed by the continuous analyte sensor during a time period that the user is adopting the posture; and controlling at least one actuator of the continuous analyte sensor system based on the compensated analyte values during the time period that the user is adopting the posture.

In an embodiment of the method, the adjunct sensor is an accelerometer. In some examples, the accelerometer may be comprised of a chip (electronic chip) that is that is attached to a transmitter board circuit included in a housing that is worn on the skin of the user, and which sits atop a location where the continuous analyte sensor is inserted into the skin of the user.

In an embodiment of the method, the adjunct sensor comprises one or more pressure sensors. In some examples, the one or more pressure sensors are coupled to an adhesive patch used to secure a housing to the skin of the user, and which sits atop a location where the continuous analyte sensor is inserted into the skin of the user.

In an embodiment of the method, the method may further comprise detecting, based at least on the first data stream and the second data stream, that the user is no longer adopting the posture. In response, the method may include providing non-compensated analyte values that accurately reflect the concentration of the analyte sensed by the continuous analyte sensor.

In an embodiment of the method, the at least one actuator may comprise an alarm configured to alert the user of an adverse event related to blood levels of the analyte. In some examples, the method may further comprise preventing the alarm from notifying the user of the adverse event provided that the compensated analyte values do not exceed one or more predetermined analyte value thresholds.

In an embodiment of the method, the analyte is glucose, and the continuous analyte sensor system is a continuous glucose monitoring system.

In an embodiment of the method, the method may further comprise retrieving data from the adjunct sensor at intervals of between 10-20 seconds.

In yet another aspect a method for a continuous analyte sensor system, comprises retrieving a first data stream corresponding to current that is reflective of a concentration of an analyte sensed by a continuous analyte sensor; converting the first data stream into analyte values reflective of the concentration of the analyte sensed by the continuous analyte sensor; retrieving one or more additional data streams from one or more additional temperature sensors positioned within a predetermined distance of the continuous analyte sensor; determining, based on the one or more additional data streams, that conversion of the first data stream is predicted to result in analyte values that do not accurately reflect the concentration of the analyte sensed by the continuous analyte sensor; and providing compensated analyte values based on the one or more additional data streams that more accurately reflect the concentration of the analyte within a predetermined threshold range of the concentration of the analyte sensed by the continuous analyte sensor.

In an embodiment of the method, the one or more additional data streams may comprise a second data stream retrieved from a first temperature sensor positioned on a transmitter board contained within a housing that is part of the continuous analyte sensor system, the housing configured to be attached to skin of the user and sit atop the continuous analyte sensor when the continuous analyte sensor is inserted into the skin of the user. In such an embodiment, providing the compensated analyte values may comprise utilizing a characterized temperature sensitivity of one or more temperature-sensitive electronic components that can adversely impact the first data stream, and temperature values corresponding to the second data stream, in a model that in turn outputs the compensated analyte values.

In an embodiment of the method, the one or more additional data streams may comprise a third data stream retrieved from a second temperature sensor positioned on a surface of the skin within the predetermined distance of the continuous analyte sensor. In such an embodiment, providing the compensated analyte values may comprise incorporating into a model that outputs the compensated analyte values a user-specific lag time corresponding to a time delay between when plasma analyte values are reflected in an equivalent change in interstitial fluid analyte levels, the user-specific lag time a function of temperature values corresponding to the third data stream.

In an embodiment of the method, the one or more additional data streams may comprise a fourth data stream retrieved from a third temperature sensor positioned on a portion of the continuous analyte sensor that is inserted into the skin of the user. In such an embodiment, providing the compensated analyte values may comprise relying on the fourth data stream to infer a diffusion rate of the analyte into the sensor, and incorporating the inferred diffusion rate into a model that outputs the compensated analyte values.

In an embodiment of the method, the analyte is glucose, and the continuous analyte system is a continuous glucose monitoring system.

In one or more or all embodiments of the method, providing the compensated analyte values is based at least in part on the current corresponding to the first data stream.

In an embodiment of the method, the predetermined distance is 2 cm or less.

In yet another aspect a method for a continuous analyte sensor system comprises retrieving a first data stream from a continuous analyte sensor configured to sense an analyte concentration in an interstitial fluid of a user; retrieving one or more additional data streams from one or more adjunct sensors positioned within a predetermined distance from the continuous analyte sensor; comparing the first data stream and the one or more additional data streams to a set of historical data that has been computationally processed to reveal patterns of data corresponding to the first and the one or more additional data streams indicative of a future event related to blood analyte levels; and providing an alert to the user that the future event is predicted to occur within a determined time frame.

In an embodiment of the method, the analyte is glucose, and the continuous analyte system is a continuous glucose monitoring system. In such an embodiment, the future event may be one of a hypoglycemic event or a hyperglycemic event.

In an embodiment of the method, the determined time frame may be between 30 minutes to 90 minutes.

In an embodiment of the method, the one or more adjunct sensors may be selected from an accelerometer, one or more temperature sensors, one or more pressure sensors, a hear rate sensor, and a blood pressure sensor.

These and other aspects of the present disclosure will become more apparent upon reading the following description.

### II. Terms

To facilitate an understanding of the embodiments herein disclosed, a select number of terms are defined below.

As used herein, the term “analyte” is to be given its ordinary and customary meaning to a person of ordinary skill in the art, and refers without limitation to a substance (e.g., chemical constituent) in a biological fluid (e.g., blood, interstitial fluid, cerebral spinal fluid, lymph fluid, urine, and the like), capable of being analyzed (e.g., in terms of concentration per specified volume). Analytes may be naturally occurring, artificial in nature, metabolites, reaction products, and the like. In a preferred embodiment, the analyte to be measured by the systems and methods of the present disclosure is glucose. However, it may be understood that the systems and methods herein disclosed apply to other analytes including but not limited to albumin, alkaline phosphatase, alanine transaminase, aspartate aminotransferase, bilirubin, blood urea nitrogen, calcium, CO₂, chloride, creatinine, glucose, gamma-glutamyl transpeptidase, hematocrit, lactate, lactate dehydrogenase, magnesium, oxygen, pH, phosphorus, potassium, sodium, total protein, uric acid, metabolic markers, acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like.

Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

As used herein, the terms “continuous analyte sensor” and “continuous glucose sensor” (also referred to as “continuous analyte monitor, or continuous glucose monitor”) are to be given their ordinary and customary meaning to a person of ordinary skill in the art, and refer without limitation to a device that continuously or continually measures a concentration of an analyte/glucose and/or calibrates the device, for example, at time intervals ranging from fractions of a second up to, for example, 1, 2, or 5 minutes, or longer.

As used herein, the term “biological sample” is to be given its ordinary and customary meaning to a person of ordinary skill in the art, and refers without limitation to a sample derived from the body or tissue of a host, for example including but not limited to blood, interstitial fluid, spinal fluid, saliva, urine, tears, sweat, and the like.

As used herein, the term “host” is to be give its ordinary and customary meaning to a person of ordinary skill in the art, and refers without limitation to animals, for example humans.

As used herein, the term “substantially” is to be given its ordinary and customary meaning to a person of ordinary skill in the art, and refers without limitation to being largely but not necessarily wholly that which is specified.

As used herein, the term “about” is to be given its ordinary and customary meaning to a person of ordinary skill in the art, and when associated with any numerical values or ranges, refers without limitation to the understanding that the amount or condition the terms modify can vary some beyond the stated amount so long as the function of the disclosure is realized.

As used herein, the terms “raw data stream” and “data stream” are to be given their ordinary and customary meaning to a person of ordinary skill in the art, and refer without limitation to an analog or digital signal directly related to the analyte concentration measured by the analyte sensor. In one example, the data stream is digital data in counts converted by an analog-to-digital (A/D) converter from an analog signal (e.g., voltage or amps) representative of an analyte concentration. The term broadly encompasses a plurality of time spaced data points from a substantially continuous analyte sensor, which comprises individual measurements taken at time intervals ranging from fractions of a second up to, for example, 1, 2, or 5 minutes or longer. As used herein, “obtaining a data stream” and “retrieving a data stream” refers to a process of acquiring the data stream from a sensor as herein disclosed via a computational device (e.g., computer) in a manner that enables the data stream to be further processed, analyzed, visualized, and the like.

As used herein, the term “counts” is to be given its ordinary and customary meaning to a person of ordinary skill in the art, and refers without limitation to a unit of measurement of a digital signal. In one example, a raw data stream measured in counts is directly related to a voltage (for example converted by an A/D converter), which is directly related to current from a working electrode.

As used herein, the term “filter” or “filtering” is to be given its ordinary and customary meaning to a person of ordinary skill in the art, and refers without limitation to modification of a set of data to make it smoother and more continuous and remove or diminish outlying points, for example, by performing a moving average of a raw data stream. In examples, filtering refers to Kalman filtering, also known as linear quadratic estimation (LQE), which relies on a Kalman filter which operates in a process that includes producing estimates of current state variables (along with their uncertainties), observing a subsequent measurement (which necessarily includes some amount of error, including random noise), and updating the estimates using a weighted average with more weight being given to estimates with higher certainty.

As used herein, the term “algorithm” is to be given its ordinary and customary meaning to a person of ordinary skill in the art, and refers without limitation to the computational processes (e.g., programs) involved in transforming information from one state to another, for example using computer processing. As used herein an “adaptive algorithm” or “learning algorithm” is to be given its ordinary and customary meaning to a person of ordinary skill in the art, and refers without limitation to an algorithm that can be trained on user specific data (e.g., current and/or historical user specific data). The adaptive algorithm can be used to ensure adjustments to a particular set of data are reflective of a particular user's physiology and/or known environmental conditions.

As used herein, the term “adjunctive sensor” is to be given its ordinary and customary meaning to a person of ordinary skill in the art, and refers without limitation to one or more sensors capable of acquiring data that is potentially relevant to data retrieved from an analyte sensor, for example a sensor capable of retrieving information related to a physiologic and/or environmental condition that can potentially impact (positively or adversely) information acquired by the analyte sensor. Relevant examples of adjunctive sensors with regard to the present disclosure include but are not limited to temperature sensors, accelerometers, pressure sensors, heart rate monitors, blood pressure monitors, and the like. As used herein, the term “adjunct sensor data” or “adjunctive sensor data” is to be given its ordinary and customary meaning to a person of ordinary skill in the art, and refers without limitation to any type of data/information that can be acquired via an adjunctive sensor. As used herein, the term “adjunctive data” need not be solely related to adjunctive sensors, but can include any adjunctive data readily attainable that relates to one or more operational aspects of the analyte sensor. Examples of adjunctive data may include but are not limited to impedance or conductivity of the user's tissues, among other parameters related to the analyte sensor (e.g., impedance of the analyte sensor itself). Since the analyte sensor analyte values are related to factors such as the user's tissue impendence and conductivity, assessing these factors could assist in making corrections to the analyte values that are displayed by the systems herein disclosed.

As used herein, the term “sensor electronics” is to be given its ordinary and customary meaning to a person of ordinary skill in the art, and refers without limitation to the components (e.g., hardware or software) of a computing device configured to process data. For example, in the case of an analyte sensor, the data may comprise biological information obtained by a sensor regarding the concentration of the analyte in a biological fluid.

As used herein, the term “operably connected” is to be given its ordinary and customary meaning to a person of ordinary skill in the art, and refers without limitation to one or more components being linked to another component(s) in a manner that allows transmission of signals between the components. For example, one or more electrodes can be used to detect the amount of glucose in a sample and convert that information into a signal. The signal can then be transmitted to an electronic circuit. In such an example, the electrode is “operably linked” to the electronic circuit. Such terms are broad enough to include wired and wireless connectivity.

As used herein, the term “sensor data” is to be given its ordinary and customary meaning to a person of ordinary skill in the art, and refers without limitation to data received from a sensor, for example a continuous analyte sensor or in other examples an adjunct sensor. Such data can include one or more time-spaced sensor data points.

As used herein, the term “potentiostat,” is to be given its ordinary and customary meaning to a person of ordinary skill in the art, and refers without limitation to an electrical system that applies a potential between the working and reference electrodes of a two- or three-electrode cell at a preset value and measures the current flow through the working electrode. The potentiostat forces whatever current is necessary to flow between the working and counter electrodes to keep the desired potential, as long as the needed cell voltage and current do not exceed the compliance limits of the potentiostat.

As used herein, the term “calibration” is to be given its ordinary and customary meaning to a person of ordinary skill in the art, and refers without limitation to the process of determining the graduation of a sensor giving quantitative measurements (e.g., analyte concentration). As an example, calibration may be updated or recalibrated over time to account for changes associated with the sensor, such as changes in sensor sensitivity and sensor background. As used herein, the term “calibration” is not meant to be the same as “compensation” or “correction” of inaccurate analyte values. As used herein, the terms “compensating” or “correcting” inaccurate analyte values are to be given their ordinary and customary meaning to a person of ordinary skill in the art, and refer to the process of providing corrected analyte values as opposed to reporting inaccurate analyte values, where the nature of the inaccurate analyte values is due to some variable impinging upon analyte sensor performance.

Compensating or correcting innacurate analyte values also broadly encompasses improving quality of data (e.g., reported analyte values) for CAM systems of the present disclosure as compared to the quality of the data in absence of the compensating or correcting. For example, lesser quality data may comprise reported analyte values that are less accurate in terms of actual concentration of analyte sensed by a continuous analyte sensor, whereas higher quality data may comprise reported analyte values that are more accurate in terms of actual concentration of analyte sensed by a continuous analyte sensor. Specifically, reported analyte values with lower accuracy may differ from the actual concentration of analyte sensed by the continuous analyte sensor by a greater extent than reported analyte values with higher accuracy.

As used herein, the term “inaccurate” with reference to reported analyte values is to be given its ordinary and customary meaning to a person of ordinary skill in the art, and refers without limitation to reported analyte values differing from actual analyte concentrations sensed by the continuous analyte sensor by some predetermined threshold amount (e.g., an inaccurate analyte value may be a reported analyte value that is outside a predetermined threshold range of the actual analyte concentration). Conversely, a reported analyte value that is accurate as herein disclosed refers to an analyte value that does not differ from the actual analyte concentration sensed by the continuous analyte sensor by more than the predetermined threshold amount (e.g., an accurate analyte value may be a reported analyte value that is not outside the predetermined threshold range of the actual analyte concentration). As used herein, the term “inaccurate analyte values” or “inaccurate values” can also refer to analyte values that are outside of some established threshold amount (e.g., outside of a threshold range) from the analyte values that would otherwise be reported in the absence of a variable impinging upon the performance of the sensor. Such variables may include but are not limited to pressure changes in the vicinity of the analyte sensor, temperature changes in the vicinity of the analyte sensor, motion-induced artifacts, and the like.

Examples of an inaccurate analyte value may be a reported analyte value that differs from the actual concentration of analyte sensed by the continuous analyte sensor (or from a reported analyte value that would otherwise be reported in absence of a variable impinging upon performance of the sensor) by >0.1%, >0.5%, >1%, or >2%, or >3%, or >4%, or >5%, or >6%, or >7%, or >8%, or >9%, or >10%, or >11%, or >12%, or >13%, or >14%, or >15%, or >16%, or >17%, or >18%, or >19%, or >20%.

Examples of such variables that impinge upon analyte sensor performance may include but are not limited to temperature effects, pressure effects, movement effects, and the like. As discussed herein, the process of providing corrected analyte values involves a process of learning situations/conditions where inaccurate values are expected or predicted to be reported, and instead of reporting the inaccurate values, providing corrected values that are based on some level of analysis of historical and/or current data trends (e.g., trends based on data retrieved from the analyte sensor and one or more adjunct sensors). For example, a sensor may be considered to be effectively calibrated, but the calibrated analyte sensor may still be prone to reporting inaccurate analyte values depending under certain select conditions as herein disclosed. In such an example, the reporting of accurate analyte values involves correcting or compensating the inaccurate values, and does not involve calibration (or re-calibration) of the sensor.

As used herein, the term “sensor session” is to be given its ordinary and customary meaning to a person of ordinary skill in the art, and refers without limitation to the period of time the sensor is applied to (e.g., implanted in) the host or is being used to obtain sensor values. As an example, a sensor session can extend from the time of sensor implantation (e.g., including insertion of the sensor into subcutaneous tissue and placing the sensor into fluid communication with a host's circulatory system) to the time when the sensor is removed.

### III. Analyte Sensor Systems and Methods of Use Sensor Systems

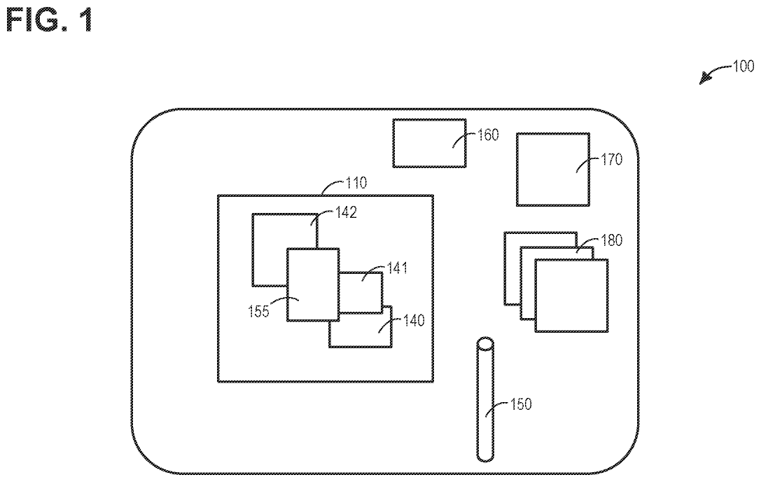

Turning to FIG. 1, depicted is a simplified diagram of a sensor system 100 (such as a CGM system) that includes a computing device, such as a computing device 110 (which can be any computing device, such as a standalone computing device), for estimating the concentration of an analyte (e.g., glucose) in tissue of a subject, for example based on an electrical signal, such as a current, from an analyte sensor 150 (e.g., glucose sensor) inserted into the tissue of a subject. Computing device 110 is broadly referred to herein as sensor electronics. With regard to the rest of the description of FIG. 1, the analyte sensor 150 is referred to as glucose sensor 150, and the analyte is referred to as glucose. The system can include glucose sensor 150 as well as one or more adjunct sensor(s), such as accelerometer 160 and temperature sensor 170. In addition to the sensors shown, the sensor system 100 can include one or more additional adjunct sensors 180. Examples of additional sensors include a blood flow monitor based on an optical evaluation of the sensor area that would help determine if there has been a change in the blood flow around the sensor that could result in lower glucose diffusion rates to the sensor or could reduce the available oxygen around the sensor. Other examples include but are not limited to a heart rate monitor, blood pressure monitor, pressure sensor(s) (e.g., potentiometric, inductive, capacitive, piezoelectric, strain gauge, variable reluctance), and the like.

In embodiments, computing device 110 includes several components, such as one or more processors 140 and at least one sensor communication module 142, for example that is capable of communication with a glucose sensor 150, accelerometer 160 and temperature sensor 170, and/or one or more additional sensors 180, for example either via a direct connection or through a signal propagated trough a transmitter and/or receiver. In various embodiments, the one or more processors 140 each include one or more processor cores. In various embodiments, the at least one sensor communication module 142 is physically and electrically coupled to the one or more processors 140. In various embodiments, the at least one sensor communication module 142 is physically and/or electrically coupled to the one or more sensors, such as a glucose sensor 150, accelerometer 160 and temperature sensor 170, and/or one or more additional sensors 180. In further implementations, the sensor communication module 142 is part of the one or more processors 140. In various embodiments, computing device 110 includes printed circuit board (PCB) 155. For these embodiments, the one or more processors 140 and sensor communication module 142 is disposed thereon. Depending on its applications, the computing device 110 includes other components that may or may not be physically and electrically coupled to the PCB. These other components include, but are not limited to, a memory controller (not shown), volatile memory (e.g., dynamic random access memory (DRAM) (not shown)), non-volatile memory (not shown) such as read only memory (ROM), flash memory (not shown), an I/O port (not shown), (not shown), a digital signal processor (not shown), a crypto processor (not shown), a graphics processor (not shown), one or more antenna (not shown), a touch-screen display (not shown), a touch-screen display controller (not shown), a battery (not shown), an audio codec (not shown), a video codec (not shown), a global positioning system (GPS) device (not shown), a compass (not shown), an accelerometer (not shown), a gyroscope (not shown) (not shown), a speaker (not shown), a camera (not shown), and a mass storage device (such as hard disk drive, a solid state drive, compact disk (CD) (not shown), digital versatile disk (DVD) (not shown), a microphone (not shown), and so forth.

In some embodiments, the one or more processors 140 is operatively coupled to system memory through one or more links (e.g., interconnects, buses, etc.). In embodiments, system memory is capable of storing information that the one or more processors 140 utilizes to operate and execute programs and operating systems, including computer readable instructions for the method disclosed herein. In different embodiments, system memory is any usable type of readable and writeable memory such as a form of dynamic random access memory (DRAM). In embodiments, computing device 110 includes or is otherwise associated with various input and output/feedback devices to enable user interaction with the computing device 110 and/or peripheral components or devices associated with the computing device 110 by way of one or more user interfaces or peripheral component interfaces. In embodiments, the user interfaces include, but are not limited to a physical keyboard or keypad, a touchpad, a display device (touchscreen or non-touchscreen), speakers, microphones, sensors, such as a glucose sensor 150, accelerometer 160 and temperature sensor 170, and/or one or more additional sensors 180, haptic feedback devices and/or one or more actuators, and the like.

In some embodiments, the computing device can comprise a memory element (not shown), which can exist within a removable smart chip or a secure digital (“SD”) card or which can be embedded within a fixed chip. In certain example embodiments, Subscriber Identity Component (“SIM”) cards may be used. In various embodiments, the memory element may allow a software application resident on the device. In embodiments, a I/O link connecting a peripheral device to a computing device is protocol-specific with a protocol-specific connector port that allows a compatible peripheral device to be attached to the protocol-specific connector port (i.e., a USB keyboard device would be plugged into a USB port, a router device would be plugged into a LAN/Ethernet port, etc.) with a protocol-specific cable. Any single connector port would be limited to peripheral devices with a compatible plug and compatible protocol. Once a compatible peripheral device is plugged into the connector port, a communication link would be established between the peripheral device and a protocol-specific controller.

In embodiments, a non-protocol-specific connector port is configured to couple the I/O interconnect with a connector port of the computing device 110, allowing multiple device types to attach to the computing device 110 through a single physical connector port. Moreover, the I/O link between the computing device 110 and the I/O complex is configured to carry multiple I/O protocols (e.g., PCI Express®, USB, DisplayPort, HDMI, etc.) simultaneously. In various embodiments, the connector port is capable of providing the full bandwidth of the link in both directions with no sharing of bandwidth between ports or between upstream and downstream directions. In various embodiments, the connection between the I/O interconnect and the computing device 110 supports electrical connections, optical connections, or both.

In some embodiments, the one or more processors 140, flash memory, and/or a storage device includes associated firmware storing programming instructions configured to enable the computing device 110, in response to execution of the programming instructions by one or more processors 140, to practice all or selected aspects of a method of estimating the concentration of glucose in tissue of a subject a sensor inserted into the tissue of a subject using a computing device, in accordance with embodiments of the present disclosure.

In embodiments, the sensor communication module 142 enables wired and/or wireless communications for the transfer of data to and from the computing device 110 for example to one or more sensors, (such as a glucose sensor 150, accelerometer 160 and temperature sensor 170, and/or one or more additional sensors 180), a transmitter, and/or transmitter/receiver coupled, such as physically and/or electrically coupled to one or more sensors, such as a glucose sensor 150, accelerometer 160 and temperature sensor 170, and/or one or more additional sensors 180.

In various embodiments, the computing device 110 also includes a network interface configured to connect the computing device 110 to one or more networked computing devices wirelessly via a transmitter and a receiver (or optionally a transceiver) and/or via a wired connection using a communications port. In embodiments, the network interface and the transmitter/receiver and/or communications port are collectively referred to as a “communication module” (e.g., communication module 142). In embodiments, the wireless transmitter/receiver and/or transceiver may be configured to operate in accordance with one or more wireless communications standards. The term “wireless” and its derivatives may be used to describe circuits, devices, systems, methods, techniques, communications channels, etc., that may communicate data through the use of modulated electromagnetic radiation through a non-solid medium. The term does not imply that the associated devices do not contain any wires, although in some embodiments they might not. In embodiments, the computing device 110 includes a wireless communication module for transmitting to and receiving data, for example for transmitting and receiving data from a network, such as a telecommunications network. In examples, the communication module transmits data, including video data, though a cellular network or mobile network, such as a Global System for Mobile Communications (GSM), General Packet Radio Service (GPRS), cdmaOne, CDMA2000, Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/TDMA), and Integrated Digital Enhanced Network (iDEN), Long-Term Evolution (LTE), 3^{rd} generation mobile network (3G), 4th generation mobile network (4G), and/or 5th generation mobile network (5G) networks. In embodiments, the computing device 110 is directly connect with one or more devices via the direct wireless connection by using, for example, Bluetooth and/or BLE protocols, WiFi protocols, Infrared Data Association (IrDA) protocols, ANT and/or ANT+protocols, LTE ProSe standards, and the like. In embodiments, the communications port is configured to operate in accordance with one or more known wired communications protocol, such as a serial communications protocol (e.g., the Universal Serial Bus (USB), FireWire, Serial Digital Interface (SDI), and/or other like serial communications protocols), a parallel communications protocol (e.g., IEEE 1284, Computer Automated Measurement And Control (CAMAC), and/or other like parallel communications protocols), and/or a network communications protocol (e.g., Ethernet, token ring, Fiber Distributed Data Interface (FDDI), and/or other like network communications protocols).

In embodiments, the computing device 110 is configured to run, execute, or otherwise operate one or more applications, such as for estimating the concentration of glucose in tissue of a subject. In embodiments, the applications include native applications, web applications, and hybrid applications. For example, the native applications are used for operating the computing device 110, sensor coupled to the computing device 110, and other like functions of the computing device 110. In embodiments, native applications are platform or operating system (OS) specific or non-specific. In embodiments, native applications are developed for a specific platform using platform-specific development tools, programming languages, and the like. Such platform-specific development tools and/or programming languages are provided by a platform vendor. In embodiments, native applications are pre-installed on computing device 110 during manufacturing, or provided to the computing device 110 by an application server via a network. Web applications are applications that load into a web browser of the computing device 110 in response to requesting the web application from a service provider. In embodiments, the web applications are websites that are designed or customized to run on a computing device by taking into account various computing device parameters, such as resource availability, display size, touch-screen input, and the like. In this way, web applications may provide an experience that is similar to a native application within a web browser. Web applications may be any server-side application that is developed with any server-side development tools and/or programming languages, such as PHP, Node.js, ASP.NET, and/or any other like technology that renders HTML. Hybrid applications may be a hybrid between native applications and web applications. Hybrid applications may be a standalone, skeletons, or other like application containers that may load a website within the application container. Hybrid applications may be written using website development tools and/or programming languages, such as HTML5, CSS, JavaScript, and the like.

In embodiments, hybrid applications use browser engine of the computing device 110, without using a web browser of the computing device 110, to render a website's services locally. In some embodiments, hybrid applications also access computing device capabilities that are not accessible in web applications, such as the accelerometer, camera, local storage, and the like. Any combination of one or more computer usable or computer readable medium(s) may be utilized with the embodiments disclosed herein. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. Note that the computer-usable or computer-readable medium can even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to wireless, wireline, optical fiber cable, RF, etc.

Computer program code for carrying out operations of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computing device, partly on the user's computing device, as a stand-alone software package, partly on the user's computing device and partly on a remote computer or entirely on the remote computer or server. In the latter scenario, the remote computer may be connected to the user's computing device, through any type of network, including a local area network (LAN) or a wide area network (WAN), or the connection may be made to an external computing device, (for example, through the Internet using an Internet Service Provider), or wireless network, such as described above.

Furthermore, example embodiments may be implemented by hardware, software, firmware, middleware, microcode, hardware description languages, or any combination thereof. When implemented in software, firmware, middleware or microcode, the program code or code segments to perform the necessary tasks may be stored in a machine or computer readable medium. A code segment may represent a procedure, a function, a subprogram, a program, a routine, a subroutine, a module, program code, a software package, a class, or any combination of instructions, data structures, program statements, and the like.

In various embodiments, an article of manufacture may be employed to implement one or more methods as disclosed herein. The article of manufacture may include a computer-readable non-transitory storage medium and a storage medium. The storage medium may include programming instructions configured to cause an apparatus to practice some or all aspects a method of estimating the concentration of glucose in tissue of a subject using a computing device, in accordance with embodiments of the present disclosure. The storage medium may represent a broad range of persistent storage medium known in the art, including but not limited to flash memory, optical disks or magnetic disks. The programming instructions, in particular, may enable an apparatus, in response to their execution by the apparatus, to perform various operations described herein. For example, the storage medium may include programming instructions configured to cause an apparatus to practice some or all aspects of a method of estimating the concentration of glucose in tissue of a subject using a computing device, in accordance with embodiments of the present disclosure.

Networked Continuous Analyte Monitoring (CAM) System

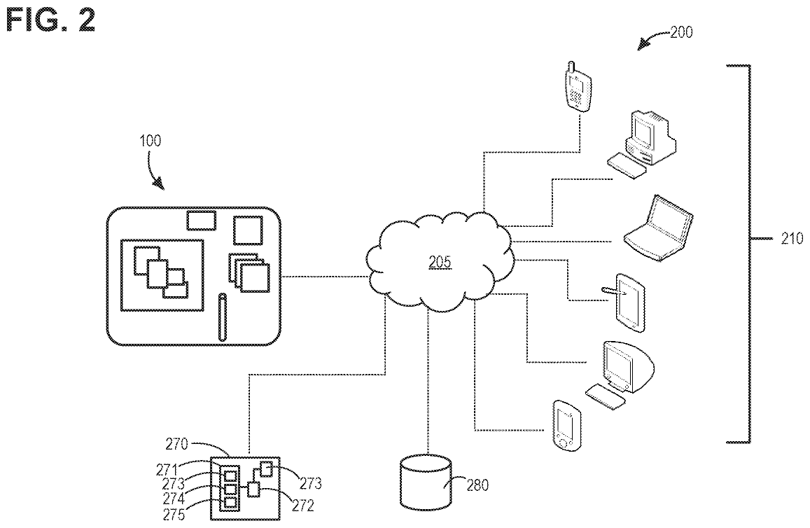

Turning to FIG. 2, illustrated is a networked CAM system 200, in accordance with embodiments herein. The networked CAM system 200 includes sensor system 100 in wireless (or wired) communication therewith. For the remainder of the description corresponding to FIG. 2, the networked CAM system is referred to as a networked CGM system. The networked CGM system 200 also includes other networked devices 210, which may be in wired or wireless communication therewith. In some embodiments, the sensor system 100 includes application software with executable instructions configured to transmit and receive information from network 205. The information can be transmitted to and/or received from another device, such as one or more networked devices 210 through a network. In certain examples, the sensor system 100 is also capable transmitting information about analyte measurements retrieved from one or more analyte sensor(s) (e.g., 150) to one or more of a doctor, other medical practitioner.

As depicted in FIG. 2, the CGM system 200 distributes and receives information to and from one or more networked devices 210 through one or more of network 205. According to various embodiments, network 205 may be any network that allows computers to exchange data, for example for cloud based storage of data generated (historical and current) and/or implementation of some, none or even all of the methods disclosed herein. Depicted at FIG. 2 is database 280, which in some examples may comprise cloud-based data storage. In some embodiments, network 205 includes one or more network elements (not shown) capable of physically or logically connecting computers. The network 205 may include any appropriate network, including an intranet, the Internet, a cellular network, a local area network (LAN), a wide area network (WAN), a personal network or any other such network or combination thereof. Components used for such a system can depend at least in part upon the type of network and/or environment selected. Protocols and components for communicating via such a network are well known and will not be discussed herein in detail. In embodiments, communications over the network 205 are enabled by wired or wireless connections, and combinations thereof. Each network 205 includes a wired or wireless telecommunication means by which network systems may communicate and exchange data. For example, each network 205 is implemented as, or may be a part of, a storage area network (SAN), personal area network (PAN), a metropolitan area network (MAN), a local area network (LAN), a wide area network (WAN), a wireless local area network (WLAN), a virtual private network (VPN), an intranet, an Internet, a mobile telephone network, such as Global System for Mobile Communications (GSM), General Packet Radio Service (GPRS), cdmaOne, CDMA2000, Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/TDMA), and Integrated Digital Enhanced Network (iDEN), Long-Term Evolution (LTE), 3^{rd} generation mobile network (3G), 4th generation mobile network (4G), and/or 5th generation mobile network (5G) networks, a card network, Bluetooth, near field communication network (NFC), any form of standardized radio frequency, or any combination thereof, or any other appropriate architecture or system that facilitates the communication of signals, data, and/or messages (generally referred to as data). Throughout this specification, it should be understood that the terms “data” and “information” are used interchangeably herein to refer to text, images, audio, video, or any other form of information that can exist in a computer-based environment.

In an example embodiment, each network system (including sensor system 100 and networked devices 210) includes a device having a communication component capable of transmitting and/or receiving data over the network 205. For example, networked device 210 may comprise a server, personal computer, mobile device (for example, notebook computer, tablet computer, netbook computer, personal digital assistant (PDA), video game device, GPS locator device, cellular telephone, smartphone, or other mobile device), a television with one or more processors embedded therein and/or coupled thereto, or other appropriate technology that includes or is coupled to a web browser or other application for communicating via the network 205.

In some embodiments where CAM system 200 is configured as a CGM system, the system may in some examples include an insulin delivery unit 270. Insulin delivery unit 270 may be comprised of at least three parts, including but not limited to insulin pump 271, tubing 272 and infusion set 273. In an embodiment, insulin pump 271 may be battery powered and may contain (or be fluidically coupled to) an insulin reservoir (e.g., container), a pumping mechanism (e.g., pump driven by a small motor) and one or more buttons and/or touch screen (not shown) to program insulin delivery. In some examples, insulin pump 271 may receive instructions for insulin delivery over network 205, from computing device 110 (refer to FIG. 1) or one of networked devices 210. The instructions may be based on analyte (e.g., glucose) concentrations as obtained via sensor system 100. In such an example, it may be understood that insulin delivery unit 270 may operate in a closed-loop fashion with other components of CGM system 200 (e.g., computing device 110 at FIG. 1 and/or one of networked devices 210) to mimic the way a pancreas works. It may be understood that each of insulin pump 271, tubing 272 and infusion set 273 may be coupled to each other in order to enable insulin pump 271 to deliver insulin to a subject by way of tubing 272 and infusion set 273. While insulin pump 271 may be battery powered, it may be understood that in some additional or alternative examples insulin pump 271 may be powered by electrically coupling insulin pump 271 to an external power source.

In some examples, insulin pump 271 may include buttons and/or a touch screen (not shown) for programming insulin delivery parameters. In another additional or alternative example, as mentioned above, insulin pump 271 may receive instructions for insulin delivery over network 205. Accordingly, in some examples insulin pump 271 may include a communication module 276 (e.g., receiver, or transceiver) capable of receiving and/or sending information (wired or wirelessly) over network 205, printed circuit board 274, and microprocessor 275. Other components of insulin pump 271 that are not shown may include one or more of a memory controller, volatile memory (e.g., DRAM), non-volatile memory (e.g., ROM), flash memory, etc.

Tubing 272 may in some examples comprise a thin tube fludically coupled to each of the insulin reservoir and infusion set 273. Tubing 272 may be plastic, teflon, etc. Infusion set 273 may comprise componentry made of teflon and/or steel, and may attach to skin of a subject by way of an adhesive patch. The infusion set 273 may include a short thin tubing (e.g., cannula) that is inserted to skin via a needle housed within the cannula. After insertion, the needle may be removed and the thin cannula may remain under the skin. It may be understood that the above description relates to an example infusion set, but other similar infusion sets may be used interchangeably without departing from the scope of this disclosure.

Methods of Use

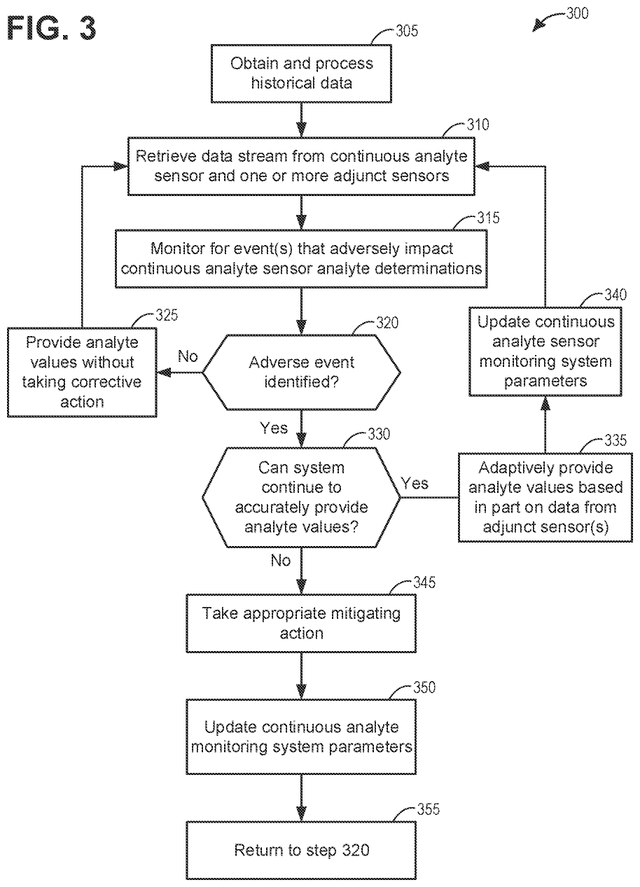

Turning now to FIG. 3, depicted is a high level example method for controlling operation of a CAM system (e.g., CGM system 200 at FIG. 2) in accordance with various embodiments. Method 300 may, at least in part, comprise executable instructions stored on a memory of, for example, a computing device (e.g., computing device 110 at FIG. 1 and/or one or more of networked devices 210 at FIG. 2). When executed, the instructions may cause a change in one or more operational states of the CGM system, for example a physical change in the manner in which insulin is delivered to a subject, a manner in which data obtained from one or more analyte sensor(s) (e.g., analyte sensor 150 at FIG. 1) and/or one or more adjunct sensor(s) (e.g., accelerometer 160 and temperature sensor 170 at FIG. 1) is used to estimate blood glucose values, a manner in which an alarm (e.g., auditory and/or vibratory) is controlled, and the like. The description below regarding method 300 is written with regard to CGM systems, but it may be understood that the methodology equally applies to other CAM systems without departing from the present disclosure.

At block 305, method 300 includes obtaining and processing historical data corresponding to one or more data streams related to use of a CGM system. Historical data as herein disclosed pertains to relevant data acquired and stored over a predetermined amount of time (e.g., 1-5 days, 5-10 days, 10-15 days, 15-30 days, 30-60 days, 60-120 days, 120-240 days, 240-365 days, or more). Relevant data includes any and all data that could be used in a learning algorithm to correlate particular data streams from sensor(s) or other acquired data/information (discussed below) with times (e.g., time periods) when glucose concentration estimates based on raw data acquired from a CGM sensor may be considered accurate or inaccurate. In embodiments, this may not be as simple as “accurate” or “inaccurate”, but may comprise a level of confidence in the glucose concentration estimates (e.g., high confidence, medium confidence, low confidence). Learning algorithms as herein disclosed pertain to, for example, artificial intelligence, and encompass subsets thereof including machine learning, deep learning, and neural networks.

Relevant data may include but is not limited to sensor data obtained from a glucose sensor (e.g., sensor 150 at FIG. 1) (or plurality of glucose sensors in a situation in which the historical data pertains to more than one sensor session); sensor data obtained from one or more adjunct sensor(s) (e.g., accelerometer 160 and temperature sensor 170 at FIG. 1); actual blood glucose measurements obtained, for example, via finger-pricking and testing of actual blood samples; and other physiological variables including but not limited to heart rate patterns, blood pressure patterns, and the like.

In some examples, relevant data additionally or alternatively includes data provided via a user. The data may be provided, for example, by a user via a software application running on a computing device of the user (e.g., networked computing device 210 at FIG. 2, such as a smartphone). Such data may include but is not limited to information pertaining to when the user is exercising (and degree of exercise such as mild, moderate, or high-intensity); form of exercise (e.g., cardiovascular, strength training, walking, etc.); when the user is in a vehicle that is traveling to a destination; when the user is sleeping; when the user is sitting/resting; when the user is working; type of food/amount of food/time of meal or snack; time of day when the user takes a prescribed medication; type and dosage of prescribed medication(s) taken by the user; time of day when the user takes one or more supplements; type and dosage of supplements taken by the user; what type of clothing the user is wearing (e.g., loose clothing, tight clothing, clothing that may create increased pressure in the vicinity of a glucose sensor, etc.), and any other variables that may be relevant to how a CGM system and associated glucose sensor acquire and process information related to glucose levels in the body of the user.

In some examples, some data may not be specifically input by the user, but may be inferred in other ways. For example, the software application running on the user's computational device (e.g., smartphone) may infer from one or more other software applications where the user currently is (e.g., geographical location, proximity to particular venue/establishment), and even what the user is currently doing (e.g., a likelihood/probability that the user is participating in a particular activity). For example, the software application associated with the CGM system may be capable of retrieving information from one or more other applications stored on the user device, to thereby infer where the user is and what activity the user is participating in. In one example, the user may rely on a radio frequency identifier (RFID) stored on the user device in order to enter into a gym. The application associated with the CGM system may retrieve such information, along with other information such as current geographical location, to infer that the user is at the gym and likely to be exercising for some time period. Another example includes an indication that the user is at a particular restaurant (e.g., retrieved from geographical location and/or social media platform in which the user has posted, for example, a picture or message pertaining to the particular dining experience). In some examples, the information can even include what type of food the user may be eating (if not input specifically by the user). An example includes an indication that the user is at an ice cream shop (based for example on location tracking, credit card statement, etc.), as opposed to a health food venue.

The sorts of data discussed above may obtained and stored, for example, at a database associated with the CGM system (e.g., database 280 at FIG. 2). In some examples, data may be obtained at regular intervals (e.g., every 1-60 seconds, every 1-5 minutes, every 5-10 minutes, every 10-20 minutes, every 20-30 minutes, every half-hour to hour, every 1-5 hours, every 5-10 hours, every 10-24 hours, every 24 hours to 2 days, and so on). For example, sensor data may be acquired more often than data pertaining to data related to user activities, meal information, and the like. The learning algorithm may reside on a memory of a user computational device (e.g., user devices 210 at FIG. 2), or in some examples may reside in the cloud or other similar database that enables the algorithm to regularly execute operations to learn patterns from the diverse types of data being acquired and stored for analysis thereof.

As mentioned above, the learning algorithm may operate on data acquired and stored for at least a predetermined amount of time. Data outside of the predetermined time period (and hence patterns learned based on said data) may be periodically forgotten, in some embodiments. In other embodiments, for example an incremental learning algorithm, the algorithm may adapt to newly acquired data without forgetting its existing knowledge. In one such example, the incremental learning algorithm may have some built-in parameter or assumption that controls the relevancy of older data.

As one example, the predetermined amount of time pertains to a predetermined number of days such as between 1 day and 365 days (e.g., between 1-2 days and 1 month, between 1-2 days and 2 months, between 1-2 days and 3 months, between 1-2 days and 4 months, between 1-2 days and 5 months, and so on). In another example, the predetermined amount of time pertains to a predetermined number of sensor sessions, for example between 1-50 sensor sessions, where a sensor session is anywhere from 1-15 days, or even higher in some examples (e.g., 15-30 days). The predetermined amount of time may comprise an amount of time in which the learning algorithm arrives at conclusions of a particular confidence level (e.g., medium-to-high confidence, or confidence of 7-10 on a scale of 1-10 where lower numbers correlate to lesser confidence). Such conclusions will be elaborated in greater detail below, but pertain to the ability to infer situations/conditions where CGM system glucose estimations are predicted to be inaccurate, as opposed to other situations/conditions where CGM glucose estimations are predicted to be accurate.

Erroneous glucose concentration estimates are highly undesirable aspects of any CGM system, but particularly in cases where the CGM system is paired with insulin pumps. Thus, the ability to computationally learn and recognize particular situations where glucose concentration estimates are inferred based on historical data analysis, represents an advantage that can be used to improve existing CGM systems as elaborated in greater detail below.

In embodiments, the historical data may not be confined to a particular individual, but may comprise population-based data. As an example, data from at least two individuals, and in some examples many more than 2 (e.g., tens, hundreds, or even thousands or more) may be fed into the learning algorithm to unearth patterns in the population-based data set. Such an approach may increase confidence in the association of particular types of data with particular events/conditions. For example, this may enable the algorithm to deduce patterns specific to particular age group, gender, race, patterns specific to users that are on a similar or same medication regimen, patterns specific to users taking a similar or same set of supplements, patterns specific to geographical location (e.g., colder climates where users may be inclined to turn up the heating in their home as compared to milder climates), and the like.

With the historical data obtained and processed at block 305, method 300 proceeds to block 310. At block 310, method 300 includes retrieving a data stream from the glucose sensor, and one or more additional data streams from one or more adjunct sensor(s). While not explicitly illustrated at FIG. 3, other types of data similar to those types mentioned above used as inputs to the learning algorithm may additionally be obtained. For example, throughout a given day, raw data (e.g., current traces) from the glucose sensor, raw data from the other adjunct sensor(s) (e.g., data retrieved from the accelerometer, temperature sensor, pressure sensor, and the like), and other optional data inputs (e.g., data input by the user into the CGM software application, data retrieved by the CGM software application from one or more other software applications) may be obtained. Raw data streams from the one or more sensor(s) may be obtained at intervals of 1-2 milliseconds-500 milliseconds, 500 milliseconds-1 second, 1-60 seconds, 1-5 minutes, 5-10 minutes, etc. In some examples, the rate at which data is acquired from one sensor may be different than the rate at which data is acquired from another sensor. For example, data may be obtained from the CGM sensor at every 30 s-5 minutes, whereas data may be obtained from the temperature sensor at less frequent time intervals (e.g., every 10-20 minutes). It may be understood that other data may be obtained when possible, for example data related to meal time and type of food ingested may only be available when the user inputs the data to the CGM software application. Such examples are meant to be illustrative an non-limiting.

At block 315, method 300 includes monitoring for events that have been learned via the learning algorithm based on the historical data, which are predicted/inferred to adversely impact CGM sensor glucose value determinations. Several examples of such events are now discussed. First, CGM sensors may be impacted by pressure applied to the area of skin to which they are attached. Pressure applied in such a vicinity may significantly impact the current relayed via the CGM sensor to the sensor electronics, and hence, the degraded signal may ultimately contribute to erroneous glucose values being displayed by the device (e.g., computational device 110 at FIG. 1 or any of networked devices 210 at FIG. 2), if the contributing event is not recognized and, where possible, compensated for. For example, pressure changes in the vicinity of a glucose sensor may result in estimates of glucose concentration dropping by as much as 80 mg/dL in a matter of just a few minutes. Of course, for glucose values that may be in a range of 90-140 mg/dL, such a drop would be cause for extreme concern if real. Even for subjects whose glucose values are in ranges exceeding that of 140 mg/dL, such a drop would still be of significant concern. This may, in an example, lead to the user taking unnecessary or even counter-productive measures to correct the situations, which in some cases could make the situation worse (e.g., ingestion of glucose to compensate could lead to a hyperglycemic event).

The issue of pressure in the vicinity of a glucose sensor leading to erroneous readings being displayed may be particularly relevant to sleep events. For example, a user of a CGM device may turn or roll during sleep in such a way that pressure is applied to the area where the sensor is located on the skin. This pressure may cause a change in the raw data signal that in turn is reported as a drop in glucose concentration. Such a drop may trigger an alarm, which could unnecessarily awaken a user thus contributing to disrupted sleep patterns, which in turn may adversely exacerbate efforts to control blood sugar. Further, similar to that discussed above, if the user believes the alarm represents a real drop in blood sugar levels, and takes mitigating action to compensate, this may lead to undesirable consequences. In a closed-loop system, intervention may automatically be taken, which can of course seriously impact a user's health. Other examples where pressure in the vicinity of the glucose sensor may result in degraded glucose values being reported include but are not limited to situations where a user is wearing tight fitting clothing (e.g., tight along the waistline near the sensor), when a user is wearing a seatbelt (e.g., automobile or plane), and the like.

As another example, a change in temperature in the vicinity of a CGM sensor may have a substantial impact on the performance of the sensor, and hence the resulting glucose values reported via the device. For example, an increase in temperature may correspond to the amount of current being communicated to sensor electronics to correspondingly increase. Such an increase may be interpreted as an increase in glucose concentration and reported as such, although the culprit is not a rise in blood glucose but instead a rise in local temperature. If not recognized and, where possible, compensated for, such an aberrant reporting of glucose values may lead to a user attempting to correct the issue by self-administering a bolus of insulin (or an insulin pump being commanded to deliver the bolus in the case of a closed-loop CGM system). This may have the undesirable effect of dramatically lowering blood glucose values, thereby risking the user entering into a hypoglycemic state, in some examples. Furthermore, if the temperature rise happens during the night while the user is sleeping, the aberrant rise in glucose may trigger an alarm, undesirably waking the user and compounding the problem of blood sugar regulation, in addition to other potential health implications stemming from poor sleep quality.

As yet another example, glucose readings from a CGM device may become aberrant during periods of significant movement of the user. Data streams corresponding to user movement may be obtained, for example, via one or more accelerometers, preferably positioned in close proximity to the placement of the CGM sensor in the user's skin. Over time, via the learning algorithm, particular patterns of movement and/or duration of said particular patterns may be learned and stored for comparison to current levels of movement. In this way, the CGM system may, based on learned movement patterns, be capable of predicting/inferring when the user is engaging in an activity that may result in inaccurate glucose readings.

It may be understood that the process of learning various events/conditions where glucose readings are expected to be accurate as opposed to inaccurate may rely on more than one type (e.g., a plurality) of data. For example, to infer that a user is sleeping, accelerometer data may be relied upon. In a case where accelerometer data shows very little to no movement, and where the pressure data indicates a sudden or gradual increase, it may be inferred that the user is sleeping and has rolled or turned into a position that applies some level of increased pressure in the vicinity of the glucose sensor. In some examples, such a determination may additionally rely on data corresponding to heart rate, blood pressure, and the like. In this way, the CGM system may increase confidence in correlating the data with a particular event.

As another example, a combination of one or more of accelerometer data, heart rate data, blood pressure data, temperature data, and even other types of data may be used to infer that a user is exercising. When combinations of data are relied upon, it may even be possible to learn what sort of exercise the user is engaging in. For example, based on learned patterns of sensor data, it may be inferred as to whether the user is engaged in mild exercise (e.g., walking), as opposed to higher intensity exercise (e.g., running, swimming, etc.). Depending on the amount of data collected, it may be possible to predict the approximate length of time that the user is engaging in a particular activity. For example, the user may go to the gym every day and engage in higher intensity workouts for a first period of time every other day, and lesser intensity workouts for a second period of time on other days. Such an example is meant to be illustrative.

Discussed herein, it may be understood that the learning methodology need not rely on any actual blood glucose readings. Instead, the CGM system may be capable of accurately predicting glucose values during particular times where it is inferred that the reported values have become erroneous, and thereby reporting the corrected values instead of the erroneous values, without the need for external input to the system in terms of actual blood glucose measurements.

Thus, at block 315, method 300 includes comparing learned patterns of data acquired from analysis of the historical data, with a current set of data being obtained via the CGM system from one or more sensor(s) and/or data input or otherwise obtained via the CGM software application. Specifically, the comparing at block 315 may enable a determination as to whether the user is engaged in some activity/situation where CGM glucose readings may be, or may become, inaccurate.

Thus, at block 320, method 300 includes indicating whether an adverse event is identified, the adverse event defined as a condition/situation/event where reported CGM glucose readings are, or may become, inaccurate. If no such event is identified, at block 325 method 300 continues to provide glucose readings without taking any compensatory action (e.g., without correcting reported glucose values). However, that is not to say that no action can be taken at block 325. For example, certain operating parameters of the CGM system may be adjusted depending on the sort of data being retrieved from the one or more sensor(s) and/or other adjunct data. As on example, in a case where the accelerometer data shows very little activity, filtering parameters may be adjusted so that less averaging may be used, and/or one or more settings associated with a Kalman filter may be changed. Other adjustments to operating parameters are within the scope of this disclosure. For example, responsive to an indication that the user is sleeping, a rate at which temperature readings, or pressure readings may be increased, or in other examples decreased, as opposed to waking hours. Method 300 then continues to retrieve data from the various sensor(s) or other adjunct data inputs, and continues monitoring for events/situations where glucose values may be inaccurate.

Returning to block 320, in response to an adverse event being identified, method 300 proceeds to block 330. At block 330, method 300 includes determining whether the system can continue to provide accurate glucose values. Specifically, at block 330, method 300 determines whether the system has enough information (e.g., learned information) to report corrected glucose values that are within some acceptable threshold of actual glucose values. For example, a user may regularly roll over onto their side during sleep, causing pressure in the vicinity of the CGM sensor to result in the reported glucose values to become inaccurate. This situation may be learned over time with high confidence, and in some examples the algorithm may have enough information to infer what the glucose readings being reported should actually be, and hence can report the corrected values rather than the inaccurate values. If, at block 335, it is determined that accurate values can be provided, method 300 proceeds to block 335, where the corrected values are reported (e.g., displayed via the CGM computing device 110 and/or displayed via one or more other computing devices 210). This may enable the CGM system to continue operating without, for example, triggering an alarm (avoiding, for example, unnecessarily waking a user), and can avoid a situation where either the user or some aspect of the CGM system (e.g., insulin pump) takes action based on inaccurate glucose values.

At block 335, in some examples the system may provide some indication that the reported values comprise corrected values, and hence should be viewed with some level of caution. For example, when corrected values are being displayed, the corrected values may flash at a predetermined rate, as opposed to not flashing for non-corrected values. In an additional or alternative example, an audible alert may be triggered to indicate to the user that the reported values comprise corrected values. In other examples, the alert may comprise the reported corrected values being of a different color than non-corrected values. The color option may be selectable via the user, for example, based on preference. For example, blue or green may be used to report non-corrected values, and red may be used to report corrected values. In some examples, some aspect of the sensor system (e.g., sensor system 100 at FIG. 1), or a user computational device (e.g., networked device 210) may vibrate in some specific pattern in response to the reported values comprising corrected values, and may vibrate in another specific pattern when the reported values cease being corrected values. Again, this feature may be user-defined.

In some examples where corrected values are reported, the system may provide some indication of a confidence level in the reported values. This may improve user satisfaction in that they may avoid anxiety over whether or not the corrected values are likely to be accurate reflections of blood glucose or not. For example, different color schemes may be used to indicate high, medium, or low confidence in corrected values. For example, non-corrected values may be blue, low confidence corrected values may be red, medium confidence corrected values may be yellow, and high confidence corrected values may be green. Such an example is meant to be illustrative. In another additional or alternative example, wording may be displayed along with reported values to indicate that the values are corrected values, of a particular confidence level. The user may be alerted in some manner (e.g., audibly, vibrationally, visually, etc.) that the reported values comprise corrected values, and then an additional layer of information may be communicated to the user as to the confidence level in the corrected values. In an example where the user is sleeping, a medium and/or high confidence in reported corrected values may prevent an alarm from being triggered that wakes the user, whereas a low confidence in reported corrected values may cause the alarm to be triggered so that a user is apprised of the potential adverse health situation.

In examples where the CGM system is operably connected to an insulin pump, there may be circumstances where the insulin pump can continue to operate using corrected values, and other circumstances where it may be preferable to discontinue any closed-loop action that involves relying on corrected analyte values to control the insulin pump (or any other aspect of closed loop operation). In one example, closed-loop operation may be maintained in situations where medium-to-high confidence, or in other examples only high-confidence, in the corrected analyte values is established. In a situation where the corrected values are determined to be of low confidence, or even medium confidence in some examples, closed-loop operation may be discontinued so that the insulin pump, for example, is not triggered to operate based on lower confidence corrected analyte values.

With regard to low confidence values, the system may over time learn the factors that contributed to the low confidence value, in the interest of converting low confidence values into medium and/or high confidence values. Specifically, the learning algorithm may be programmed to learn/assess what type of event led to the low confidence corrected values, and based on other situations where higher confidence corrected values are reported, the system may over time be capable of increasing confidence in the corrected values reported for an event that was previously correlated with corrected glucose values of low confidence level.

Responsive to the providing of corrected values, method 300 continues to block 340, and includes updating CGM system parameters based on the event that led to the providing of corrected values. Updating CGM system parameters may include but is not limited to storing additional data retrieved from any of the glucose and/or adjunct sensor(s), storing an indication that corrected values were provided for a particular duration of time, storing any actual blood glucose values inputted into the system during and/or following the event where corrected values were displayed, updating any relevant filtering parameters (e.g., filtering parameters may be changed during a particular adverse event, and then may be changed back or otherwise updated once the event has passed), and the like. It may be understood that any and all of the above-mentioned updates to CGM system parameters may comprise data that can be fed back into the learning algorithm to enable the algorithm to continue to improve its ability to accurately assess situations where reported glucose values may be inaccurate, and, where possible, provide corrected glucose values of higher and higher confidence.

Retuning to block 330, it is herein recognized that there may be circumstances where the system determines that it is not capable of accurately providing corrected glucose values. There may be any number of reasons why this may, in some examples, be the case. As one example, the adverse event may comprise an event that is similar to other adverse events learned over time, but with a certain level of difference that does not enable accurate determination as to what the corrected glucose values should be. As another example, the learning algorithm may not yet have processed enough information, or have been feed enough data, to accurately predict corrected glucose values. In some examples, there may be some possibility of adjunct sensor (or even glucose sensor) degradation, that is confounding the ability to accurately assess what sort of event is actually occurring. As one particular example, a sudden temperature drop without some other explanation during sleeping hours, or during exercise, may be indicative of a degraded temperature sensor, but also could have other potentially serious health implications. Such an example is not meant to be limiting, but illustrative in nature. It may be understood that, under all circumstances, health of the user is the top priority, and hence if there is any indication that corrected glucose values may not accurately reflect the underlying biology, then other mitigating action may be taken.

Specifically, at block 345, method 300 includes taking mitigating action. The mitigating action may comprise an alarm/alert (e.g., visual, auditory, vibrational, etc.) communicated to the user that reported CGM values cannot currently be trusted. In some examples, rather than displaying any reported values, the system may instead display an error message, or other message that communicates to the user the fact that glucose values as determined via the CGM system are currently compromised. The error message may flash, for example. In such a situation, the user may be informed that it would be in their best interest to use some other means of assessing current blood glucose levels. For example, the system may display a message requesting the user to rely on actual blood glucose readings for a certain determined amount of time. It may be understood that these actual blood glucose readings may be in turn stored and, in some examples, used as additional data in the learning algorithm.

In the case where the system cannot continue to provide accurate analyte values (e.g., confidence in the analyte values is low, or even well below what is considered low with regard to block 335), any closed-loop operation may be discontinued, and the user may be alerted to this fact. For example, reliance on the insulin pump may be discontinued, and any action that needs be taken to control blood sugar may have to be taken manually by the user. The user may then be alerted to when closed-loop operation resumes, so that the user is apprised of this information thus avoiding a situation where the user continues to manually address blood sugar control.

As mentioned, in some examples the adverse event may be caused by some level of degradation of a particular sensor, leading to what appears to be an adverse event but which may in fact, be solely due to sensor degradation. In some examples, taking mitigating action at block 345 may comprise the system requesting an action by the user that in turn, enables the system to assess whether one or more sensor(s) are operating as expected or desired. For example, the system may infer that it is possible that a pressure sensor has become degraded. Hence, the system may issue a request to the user to apply pressure in the vicinity of the pressure sensor, and this may enable the system to assess whether the pressure sensor is operating as expected or not. For example, the user may input into the system that they are about to apply pressure, and may confirm once the pressure has been applied (or immediately after). The CGM system may then assess whether the pressure sensor responded as expected or not, and this information may be used to determine the potential for pressure sensor degradation. In the event that the pressure sensor is indicated to be degraded, the CGM system may issue a request to the user to replace the pressure sensor. The user may input to the system confirmation that the sensor has been replaced once the action has been taken.

Similar examples apply to other sensors. For example, responsive to an indication that the accelerometer may be performing erratically, the CGM system may request the user to perform some predetermined sequence of motion (e.g., bend over and straighten up 1-3 or more times, walk in a circle or square of approximate dimensions, and the like). With regard to the temperature sensor, the user may be requested to apply some form of heat or cold to the vicinity of the sensor (e.g., hot or cold washcloth, etc.) to assess whether the temperature sensor responds as expected. Other examples are within the scope of this disclosure. Similar examples apply to other types of sensors, including but not limited to heart rate monitors, blood pressure monitors, etc. For example, the system may request an alternative means of determining heart rate or blood pressure, that can then be input into the CGM system to enable a determination as to whether a particular monitor is exhibiting degraded operation, or not.

At block 350, method 300 includes updating CGM system parameters. For example, updating system parameters at 350 may include storing any data relevant to the current event, including but not limited to data retrieved from one or more of the CGM sensor and/or adjunct sensor(s), duration of time the adverse event occurred, whether any sensor(s) needed replacing and/or whether any sensor(s) were replaced, any additional blood glucose readings obtained and inputted into the system, updating any relevant filtering parameters, and the like. It may be understood that any and all of the data corresponding to the updated system parameters may be fed into the learning algorithm in order to enable the algorithm to continue to improve its ability to accurately assess situations where reported glucose values may be inaccurate, and, where possible, provide corrected glucose values of higher and higher confidence. Method 300 may then return to step 320 of method 300.

While not explicitly illustrated at FIG. 3, it is herein recognized that an ability to predict/infer when analyte values may be inaccurate, and by extension, when analyte values may be highly accurate, may be advantageous in terms of designating particular time periods for conducting analyte sensor calibration operations. For example, any calibration operation conducted during a timeframe in which analyte values may not be accurate, even if such values can be corrected for in terms of displaying to the user, may degrade the effectiveness of the calibration operation. Thus, encompassed by this disclosure is methodology for predicting time periods of sensor operation where analyte values are predicted to be accurate and not in need of any compensation, and scheduling a calibration operation at a time encompassed by said predicted time periods. The predicting of such time periods may be based on the learned patterns of sensor operation derived from analysis of historical data, as herein disclosed. However, it is herein recognized that in some examples calibration operations may be capable of being conducted during times when corrected glucose values are being relied upon, for example when the corrected glucose values are of a certain confidence level (e.g., high).

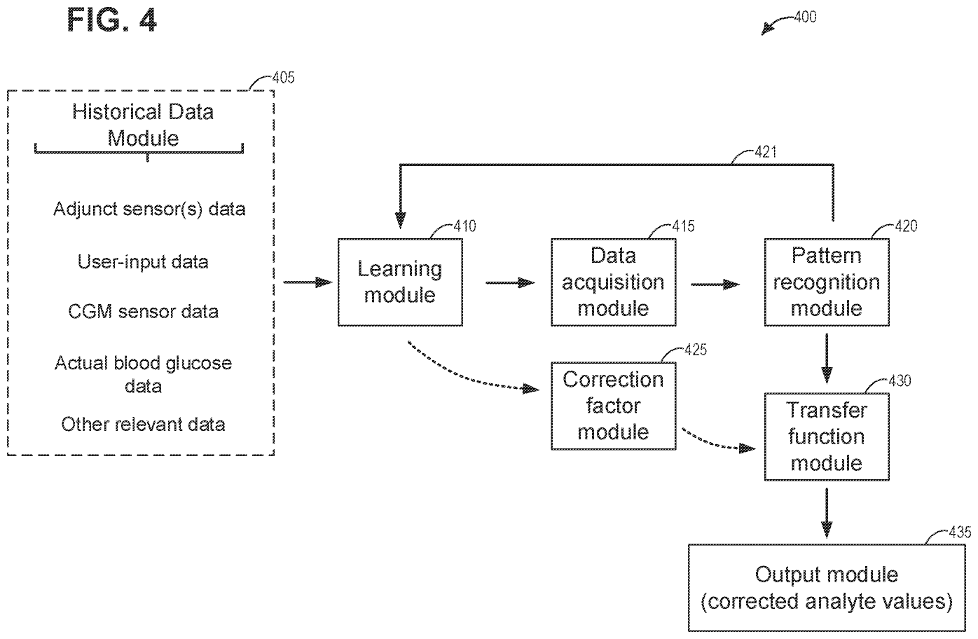

Turning now to FIG. 4, depicted is a high-level process flow 400 applicable to the method discussed above at FIG. 3. Illustrated is historical data module 405, learning module 410, data acquisition module 415, pattern recognition module 420, correction factor module 425, transfer function module 430, and output module 435. It may be understood that the process flow 400 broadly encompasses the learning algorithm discussed above at FIG. 3. For example, each module shown at FIG. 4 may comprise a subset of the learning algorithm. However, it may be understood that additional modules, or lesser modules, are within the scope of this disclosure.

Briefly, the historical data module stores any and all relevant historical data for predicting/inferring times when the CGM system may potentially report inaccurate glucose values. That data may include, but is not limited to, data acquired from adjunct sensor(s), data input via a user into, for example, a software application operably linked with the CGM system, CGM sensor data acquired via a CGM sensor currently implanted and/or prior CGM sensor(s) already used, prior actual blood glucose measurements (along with relevant corresponding data such as time of day, day of week, time of measurement with respect to meal/snack, and the like), and any other relevant data. The other relevant data can include, for example, data that the software application has inferred based on geographical location or other information obtained from other software applications. In some examples, the historical data corresponds to a single user, but it is within the scope of this disclosure that the historical data not be limited to a single user, but rather, to a population of users.

The historical data module 405 supplies the learning module 410 with the historical data contained therein. The learning module 410 relies on some form of artificial intelligence to deduce patterns in the historical data, particularly patterns whereby it may be possible to predict with high accuracy circumstances when the CGM sensor has become unreliable (e.g., likely to report inaccurate glucose values). In examples, the learning module 410 relies on machine learning, which can comprise supervised learning, unsupervised learning, reinforcement learning or some combination thereof. The learning module 410 may, in addition to learning circumstances in which the reported glucose sensor values may be inaccurate, be programmed to predict what the glucose values actually should be, during times when the reported values have become inaccurate. Specifically, learning module 410 may feed data into the correction factor module 425 so that appropriate correction factors can be determined for various situations where the reported glucose values are predicted to be inaccurate. In some examples, the correction factor module 425 is thus a part or subset of learning module 410. There may be different correction factors that apply to different circumstances. In some examples, a same correction factor may be relied upon for a plurality of (e.g., more than one) different circumstances where reported glucose values are otherwise predicted to be inaccurate. The correction factor may be used to compensate the error in the otherwise reported glucose values, so that instead, more accurate glucose values are communicated to the user. Specifically, the correction factor may be used so that reported glucose values are within some acceptable range of what the reported glucose values should be (in the absence of the confounding circumstance/condition that renders the values inaccurate).

The data acquisition module 415 may be understood to be capable of retrieving newly acquired data from the CGM system, for use in the process flow 400 of FIG. 4. Thus, the data acquisition module is operably linked with the CGM system, capable of obtaining (e.g., in real-time) data obtained from one or more sensors (e.g., CGM sensor and/or adjunct sensor(s)) data input into the CGM software application, and any other relevant data input to the CGM system.

The pattern recognition module 420 relies on the information learned by way of the learning module 410, in conjunction with the data acquisition module 415 comprising newly acquired data, to predict/infer whether a current situation is one where it is expected that reported glucose values have become inaccurate, or not. There may be varying levels of what is meant by “inaccurate.” For example, some situations may result in reported glucose values being inaccurate by a first amount, other situations may result in reported glucose values being inaccurate by a second amount, other situations may result in reported glucose values being inaccurate by a third amount, and so on. For example, the first amount may be lesser than the second amount, which in turn may be lesser than the third amount. Hence, the correction factor module 425 may necessarily generate different correction factors for a variety of learned circumstances, as mentioned above. Furthermore, in examples, the pattern recognition module may include some estimation of probability that data newly acquired via the data acquisition module 415 corresponds to a situation where reported glucose values may be inaccurate (or accurate). This probability/likelihood may impact some aspects of method 300 as discussed with regard to FIG. 3, for example in terms of assessing whether or not compensated/corrected glucose values can be accurately reported to a user and/or with what level of confidence the user should assume the corrected glucose values correspond to.

Arrow 421 depicts the process flow as returning to learning module 410. This is meant to imply that newly acquired data, and its relationship to predetermined data patterns consistent with circumstances where reported glucose values may be inaccurate (or vice versa, where reported glucose values are likely accurate), may be fed back into the learning module 410. In this way, the learning module may be continually updated with newly acquired data and the relationship of the newly acquired data to previously established data patterns, which may improve operation of the overall process flow 400 of FIG. 4 over time.

The transfer function module 430 comprises a function (e.g., mathematical function) that translates inputs fed into the module into an output via the output module 435. The output in this example refers to glucose values, which may be understood to, at least in some circumstances, comprise glucose values that have been corrected/compensated to at least some degree as compared to glucose values otherwise reported in absence of the process flow 400 of FIG. 4. As depicted, the transfer function module 430 may receive input from the correction factor module 425, meaning that the transfer function module 430 is capable of being modified via one or more correction factors as determined via the correction factor module 425. In this way, accurate glucose values may be output via the output module 435, for a variety of circumstances in which the reported glucose values would otherwise be inaccurate to some degree. The output module 435 may output the glucose values to, for example, a display associated with the CGM computing device (e.g., computing device 110 at FIG. 1) and/or a display associated with a user computational device (e.g., one of networked devices 210 at FIG. 2), for example by way of the CGM software application.

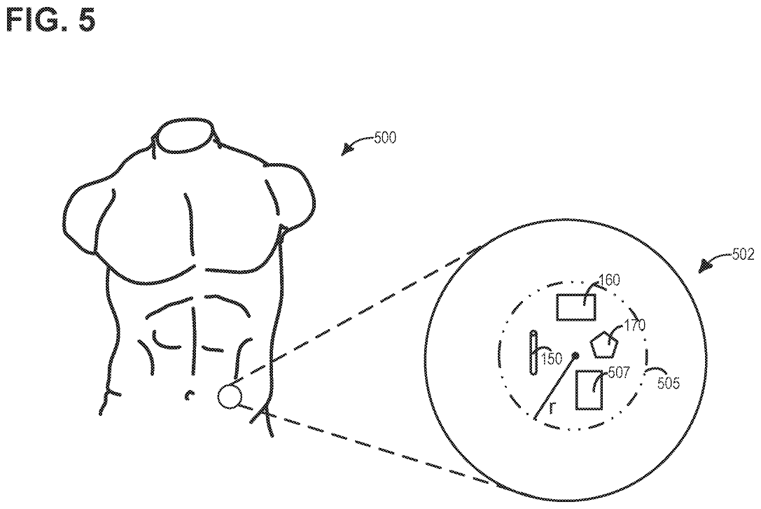

Turning now to FIG. 5, depicted is an illustration of a torso 500 of a user of a CAM system of the present disclosure. It is herein recognized that it may be advantageous for one or more adjunct sensors to be in a certain proximity to the analyte sensor. Accordingly, inset 502 shows a close-up view of a location on the torso 500 of the user where the analyte sensor 150 is embedded in the skin of the user. A region 505 of radius r defines an area where at least one other adjunct sensor is positioned. Illustrated is accelerometer 160, temperature sensor 170, and pressure sensor 507. In examples, radius r is 8 cm or less, for example 7 cm or less, 6 cm or less, 5 cm or less, 4 cm or less, 3 cm or less, 2 cm or less, or even 1 cm or less (e.g., within 1-10 mm, 10-50 mm, 50-100 mm, 100-500 mm, 500-1000 mm). Not shown at FIG. 5 is a housing that houses sensor electronics, for example a housing that includes computing device 110. Also not shown at FIG. 5 is an adhesive patch that may comprise a backing of such a housing, and which may be used to adhere the housing to a skin of the user. As elaborated below, in some examples it is within the scope of this disclosure that one or more pressure sensors 507 may be incorporated into such an adhesive patch, and these one or more pressure sensors may comprise adjunct sensors capable of reporting on pressure changes in the close proximity of the analyte sensor. In some examples, it is within the scope of this disclosure that one or more adjunct sensors including but not limited to accelerometer 160, temperature sensor 170, and pressure sensor 507 may be included within or coupled to (e.g., positioned on an external face of) such a housing. In examples, one or more of the adjunct sensors are operably linked to the electronics corresponding to the sensor electronics. In this way, the sensor electronics capable of receiving and transmitting information pertaining to the analyte sensor 150 may similarly be capable of retrieving and transmitting information pertaining to any of the operably linked adjunct sensors. In other examples, it is within the scope of this disclosure that the one or more adjunct sensors comprise stand-alone sensors, each independently capable of retrieving and transmitting data independent of any operable linkage to the sensor electronics. In examples, the one or more adjunct sensor(s) are attached to the skin of the user. In examples, the accelerometer may be integrated into sensor electronics that operates the potentiostat of the CGM device. In still other examples, one or more adjunct sensors may be positioned on a transmitter board included within the housing (not shown). For example, as elaborated in greater detail below, in some embodiments a temperature sensor may be positioned on the transmitter board.

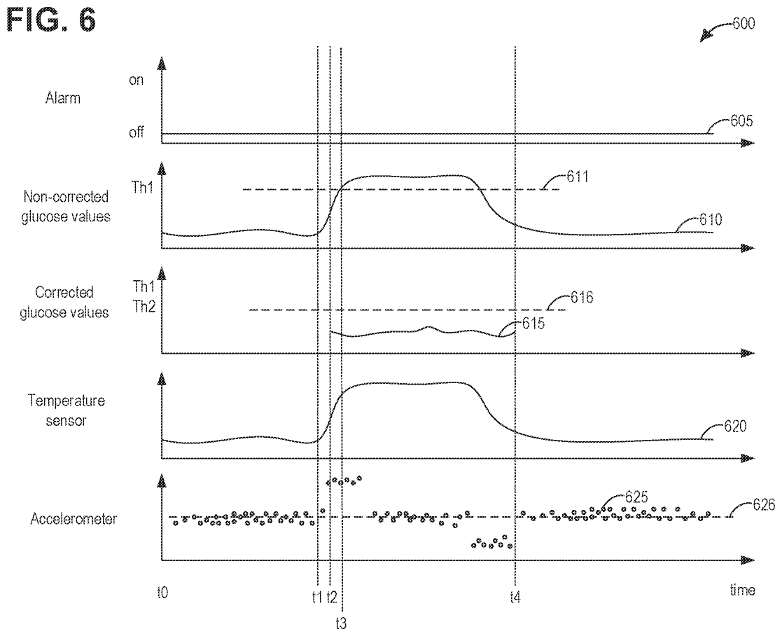

FIG. 6 depicts an example timeline 600 illustrating how an actuator of a CGM system of the present disclosure may be controlled at times when glucose values being reported to a user (for example via a display device) correspond to corrected glucose values. In this example timeline, the actuator comprises an alarm (e.g., audible or vibrational, etc.) which can be actuated in response to glucose values (corrected or non-corrected) exceeding some predetermined threshold (e.g., hyperglycemic or hypoglycemic threshold). Timeline 600 includes plot 605, indicating whether the alarm is off (e.g., deactivated), or on (e.g., activated), over time. Timeline 600 further includes plot 610, indicating non-corrected glucose values, and plot 615, indicating corrected glucose values, over time. Timeline 600 further includes plot 620, indicating data corresponding to temperature recorded via an adjunct temperature sensor, over time. Timeline 600 further includes data points 625, corresponding to accelerometer data collected over time. Line 626 reflects “no movement” respective to accelerometer data.

Between time t0 and t1, the CGM system is relying on non-corrected glucose values. There are very little changes in temperature sensed by the temperature sensor, and there is very little detectable movement associated with the user. Accordingly, between time t0 and t1, the system predicts that the non-corrected values reflect accurate representations of glucose concentration sensed by the continuous glucose sensor, within some predetermined threshold range.

Just after time t1, temperature begins increasing (plot 620), and this increase in temperature is associated with some aspect of movement, as indicated by the accelerometer data (plot 625). Based on at least the accelerometer data and the temperature data, and a comparison of this data to historical data as discussed above, the system predicts that an event is occurring which is expected to result in inaccurate glucose values that are not reflective of the actual glucose concentration sensed by the continuous glucose sensor. The system may also take into account other variables, such as time of day, for example to infer whether the user is likely sleeping, or in a vehicle, etc. As can be seen between time t1 and t2, non-corrected glucose values begin rising concomitant with the apparent movement and increased temperature as reported by the respective sensors.

Because the system is able to predict that the rise in glucose is likely artificially caused, for example, by the user rolling over in their sleep and perhaps covering themselves with a heavy blanket, thus resulting in increased temperature in the vicinity of the glucose sensor, at time t2 the system stops relying on non-corrected glucose values (plot 610), and instead begins relying on corrected glucose values (plot 615). In this example timeline, non-corrected glucose values continue to be illustrated during times then the system is relying on corrected glucose values, for reference. However, in some examples, non-corrected values may continue to be determined even during times when corrected values are being used. This may enable a comparison between corrected and non-corrected values, so that when corrected and non-corrected values are within a predetermined threshold from one another (e.g., when values are within 1-5% of one another, for example 2% of one another), the system may revert to relying on non-converted glucose values.

At time t3, if corrected values were not being relied upon, then the a first glucose value threshold (Th1, represented by line 611) would be exceeded. This would trigger the alarm to be actuated. In a situation where the user is sleeping, this would wake the user, and potentially cause the user to take inappropriate action to manage the presumed condition. However, because corrected values are being relied upon at time t3, the alarm is not activated (plot 605).

Between time t3 and t4, corrected glucose values remain below a second glucose value threshold (Th2, represented by line 616). In this example timeline 600, the Th2 threshold is lower than the Th1 threshold, although both thresholds relate to when to actuate the alarm. The Th2 threshold is lower because corrected glucose values are being used, which may comprise a confidence level less than those of non-corrected glucose values due to the computational operation(s) associated with providing corrected glucose values. To prioritize health of the user, the Th2 threshold may be lower than the Th1 threshold to bias the system toward detecting any condition which may impact the health of the user. In other words, the Th2 threshold represents a more conservative threshold than the Th1 threshold because the system is relying on corrected glucose values.

Just before time t4, there is detectable movement (data points 625) and temperature as sensed by the temperature sensor (plot 620) begins decreasing. In this example timeline 600, it may be understood that this correlates with the user rolling over once again, liberating the glucose sensor from the environment that was causing the elevated temperature.

At time t4, the system predicts that non-corrected glucose values are expected to accurately represent actual glucose concentration sensed by the continuous glucose sensor. Hence, at time t4, the system reverts to relying on non-corrected glucose values.

The discussion with regard to example timeline 600 illustrated an example where glucose value thresholds for setting the alarm were adjusted depending on whether corrected or non-corrected glucose values were being relied upon. In other examples, the thresholds may not be adjusted between the two conditions, without departing from the scope of this disclosure.

Example timeline 600 illustrated just two adjunct sensors (temperature and accelerometer), but it may be understood that any number of other adjunct sensors and relevant data to determining time periods where conversion of raw data obtained from the continuous glucose sensor are expected to be inaccurate.

Furthermore, while example timeline 600 depicts the manner in which an alarm is controlled according to embodiments of the present disclosure, in other embodiments the actuator may comprise, for example, an insulin pump included in a closed-loop CGM system. Similarly, the insulin pump may be controlled based on corrected values at times when non-corrected values are predicted to be inaccurate, according to similar logic as that depicted by the timeline of FIG. 6.

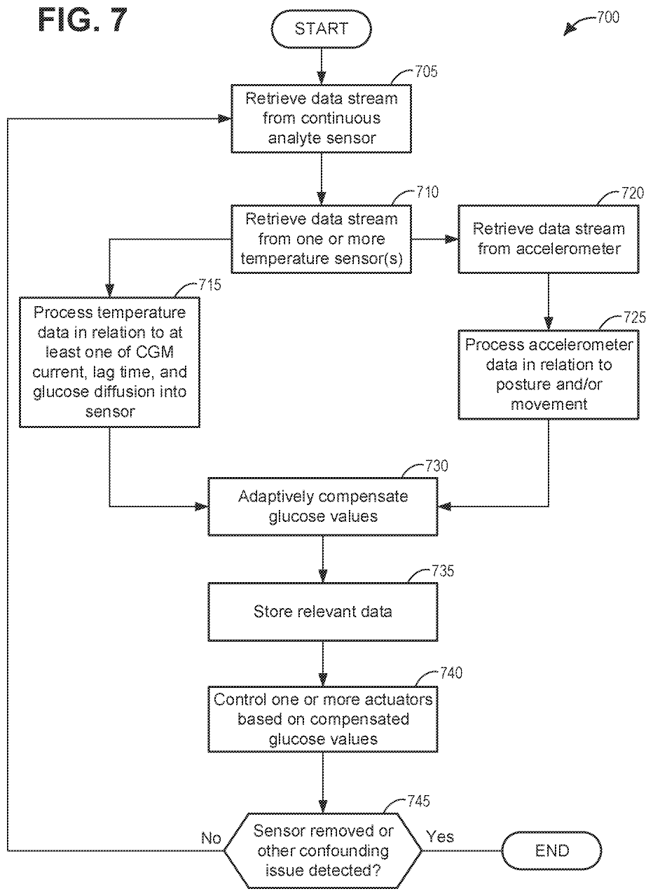

The above description is directed towards the use of adjunct sensor data in combination with a continuous analyte sensor, to infer/predict a number of scenarios where glucose values may be incorrectly reported if not adaptively corrected for. It is herein recognized that adjunct sensor data in combination with data retrieved from a continuous analyte sensor may additionally or alternatively be relied upon to improve quality of continuous analyte sensor data in other ways discussed below with regard to the methodology of FIG. 7.

FIG. 7 depicts a high-level example method for improving quality of data reported to a user and/or relied upon to control one or more actuators of a CAM system (e.g., CGM system 200 at FIG. 2), in accordance with various embodiments. Method 700 may, at least in part, comprise executable instructions stored on a memory of, for example, a computing device (e.g., computing device 110 at FIG. 1 and/or one or more of networked devices 210 at FIG. 2). When executed, the instructions may cause a change in one or more operational states of the CGM system, for example to control one or more actuators (e.g., vibratory and/or auditory alarm, insulin pump, and the like) of the CGM system. Method 700 is written with regard to CGM systems, but it may be understood that the methodology equally applies to other CAM systems without departing from the scope of this disclosure.

Method 700 begins at block 705, and includes retrieving a data stream from a CGM sensor (e.g., analyte sensor 150 at FIG. 1). Method 700 may start upon insertion of a CGM sensor into skin, for example.

Proceeding to block 710, method 700 includes retrieving a data stream from one or more temperature sensor(s). Where the CGM system includes more than one temperature sensor, it may be understood that block 710 includes retrieving a separate data stream (e.g., first, second, third, etc.) from each respective temperature sensor. Temperature data may be retrieved at regular intervals, for example at intervals between 1 second (or less) and 10 minutes. For example, temperature data may be retrieved at intervals between 1-5 seconds, between 5-10 seconds, between 10-20 seconds, between 20-30 seconds, between 30-40 seconds, between 40-50 seconds, between 50-60 seconds, between one minute and two minutes, between two minutes and three minutes, between three minutes and four minutes, between four minutes and five minutes, or between 5 minutes and 10 minutes. In some examples, temperature data from at least one sensor is obtained at intervals comprising 50-70 seconds, for example 60 seconds. This may conserve power and save on computational storage space for the CGM system, while also providing sufficient temperature data for method 700. In some examples where data from more than one temperature sensor is retrieved, the intervals between retrieving data may be the same for each temperature sensor, however in other examples the intervals may be different for different sensors.

In one example, a temperature sensor may be located on a transmitter board of, for example a computing device (e.g., computing device 110 at FIG. 1) operably linked with the CGM sensor. One advantage herein recognized to positioning a temperature sensor on the transmitter board is that the temperature sensor may be quite close to the user's body and the CGM sensor, for example when the housing (which houses the transmitter and associated computing device) is positioned on the skin of a user.

In another additional or alternative example, a temperature sensor may be positioned under the transmitter housing and in direct contact with skin. In such an example, the housing may include some vents (e.g., openings, outlets, holes, gaps, apertures, etc.) such that situations may be avoided where a lack of venting results in increased temperatures that are not accurate reflections of actual skin temperature.

In yet another additional or alternative example, a temperature sensor may be positioned on the skin of a user, within, for example 2 cm of the CGM sensor, but not between the transmitter housing and the skin.

In still another additional or alternative example, a temperature sensor may be positioned on the surface of the CGM sensor, such that the temperature sensor is inserted into the skin along with the CGM sensor upon sensor insertion.

In some examples, the CGM system has just one temperature sensor positioned at any one of the above-mentioned sites, however in other examples the CGM system may include any number of temperature sensors at two or more of the above-mentioned sites, for example three sites, or even four sites. In one particular example, the CGM system includes three temperature sensors, one positioned on the transmitter board, one positioned on the skin (either between the housing and the skin or just outside the housing), and one positioned on the CGM sensor inserted into the skin of a user.

An advantage of positioning the temperature sensor at the transmitter is that the transmitter includes a number of electronic components, each of which may be susceptible to temperature. For example, a resistor (e.g., mega ohm resistor) associated with the transmitter may be susceptible to temperature changes. If the temperature of such a resistor changes significantly, this may affect the overall function of the CGM system, for example by adversely impacting a current reading being tracked via the computing device of which the transmitter is a part of (or operably linked to). The current reading being tracked ultimately is converted to a glucose value, hence small changes in resistor characteristics, due to temperature changes thereof, may result in changes in the determined glucose values. By providing a capability to measure temperature at the transmitter board, changes in temperature may be measured and correlated with temperature sensitivity of the corresponding electronics (the temperature sensitivity previously characterized), such that any changes due to temperature can be compensated for such that the quality and accuracy of the glucose value reported may be improved.

With regard to temperature of the skin, skin temperature is a reflection of the amount of blood that is circulating therethrough. The more capillary blood that is circulated through skin, the better the equilibrium is between plasma glucose and interstitial fluid glucose. Because CGM sensors of the present disclosure measure interstitial fluid glucose, the better the equilibrium between plasma and interstitial fluid glucose, the closer the measurement of interstitial fluid glucose may be to actual blood glucose.

Furthermore, skin temperature may impact lag time, which herein refers to a time difference between when a change in plasma glucose is fully reflected in an equivalent (or approximately equivalent, for example within 1% or less, or 5% or less, or 10% or less) change in the interstitial fluid glucose, and hence, glucose concentration reported via a CGM sensor/system of the present disclosure. This lag time may vary between individuals, but may be understood to in general be somewhere between 2 and 7 minutes (although lesser and greater lag times are not outside the scope of this disclosure). The lag time may be dependent on a magnitude of change in plasma glucose levels, in some examples. Another variable that may impact lag time is blood circulation in the skin, which as mentioned above is a reflection of skin temperature. For example, colder skin temperatures may be associated with lower blood circulation, which in turn may increase the lag time. Alternatively, higher skin temperatures may be associated with greater blood circulation, which in turn may decrease the lag time. Thus, it is herein recognized that such temperature data may be incorporated into an algorithm that enables the CGM value to be compensated based on the recorded skin temperature and as a function of determined lag time. This may decrease variability in terms of the impact that skin temperature has on the lag time, thereby improving the quality and/or accuracy of the CGM values reported to the user and/or relied upon for controlling one or more operational aspects of the CGM system. It may be understood that, because lag time may be user-specific, the lag time as a function of skin temperature for specific individuals may have to be empirically learned (e.g., via a learning algorithm such as those disclosed herein), or otherwise obtained, to be effective. As an example, such a compensation may be comprised of a number of parts, including but not limited to rate of glucose change as measured by the CGM, skin temperature as retrieved via the temperature sensor, and modeled lag time characteristics as a function of skin temperature for an individual user.

Still further, skin temperature in the vicinity of the CGM sensor may have an impact on glucose diffusion into the sensor. For example, CGM sensor glucose measurements are based on measuring the glucose molecules that diffuse into the sensor, and following this diffusion into the sensor, the glucose reacts with an enzyme (e.g., glucose oxidase) to produce, for example, hydrogen peroxide. Hydrogen peroxide is then oxidized by the sensor working electrode to generate a current reflective of the concentration of glucose in the interstitial fluid. Diffusion of glucose into the sensor becomes steady-state, and by measuring the steady state at a particular glucose concentration, the glucose concentration in the interstitial fluid may be estimated. These diffusional characteristics of glucose into the sensor may be impacted by temperature. At lower temperature, diffusion rates of glucose into the sensor may be lower, thus at a lower temperature reported glucose values may be lower than the actual concentration of glucose in the interstitial fluid. As a representative example, a 5° C. shift in temperature in the vicinity of the sensor may have an impact of up to 10-12% in terms of reported glucose values. By providing a measurement of skin temperature, this data can be incorporated into an algorithm that accounts for modeled glucose diffusional characteristics as a function of temperature, so that the glucose values can be compensated to more accurately reflect the actual interstitial fluid glucose concentration sensed by the CGM sensor. Preferably, for measuring the temperature effect that contributes to diffusional characteristics of glucose, the temperature sensor is positioned on the CGM sensor inserted into the skin of a user. In other words, the temperature sensor may be inserted into the skin (not just remaining on the surface of skin but penetrated into the skin), along with insertion of the CGM sensor into skin.

In some embodiments, a CGM system of the present disclosure may include just one of the above mentioned temperature sensors, for example just the temperature sensor positioned on the transmitter board, just the temperature sensor positioned on the surface of the skin, or just the temperature sensor positioned on the CGM sensor inserted into the skin of a user. In other examples, temperature sensors may be included at more than one of the above-mentioned temperature sensor locations, for example two locations, or even all three. In the case where a CGM system of the present disclosure includes a plurality of temperature sensors, this may enable a plurality of temperature-based corrections to improve the quality and/or accuracy of the CGM sensor.

Accordingly, at block 715, method 700 includes processing the temperature data retrieved from the one or more temperature sensors. As discussed, this may be done by way of a model that accounts for particular variables associated with temperature effects, for example the manner in which electronics temperature impacts CGM current, the manner in which skin temperature impacts lag time, and the manner in which skin temperature impacts glucose diffusional characteristics into the sensor. In some examples, separate models (e.g., algorithms) may be used for each different temperature sensor, or a single model that accounts for each stream of temperature data may be used.

Returning to step 710, in some examples an accelerometer may be additionally included in the CGM system. Accordingly, at block 720, method 700 may include retrieving a data stream from the accelerometer. In a preferred example, the accelerometer may be attached to the CGM transmitter board circuit, enabling data to be collected in three axes (e.g., x, y, z). It is herein recognized that including the accelerometer in a position where it is attached to the CGM transmitter board circuit may provide unique advantages as compared to other systems which may rely on accelerometers positioned, for example, on a wrist of the user. For example, including the accelerometer at the site where the sensor is located enables data collected from the accelerometer to be precisely correlated with particular impacts on CGM sensor signals.

In one example, accelerometer data may be obtained from a user, stored, and analyzed with respect to what the user was doing (e.g., particular activity) at a particular time. This combination of data may enable the ability to correlate particular accelerometer data trends with particular user postures (e.g., bending over to tie ones shoes), and may be further correlated with relatively brief (e.g., less than 5 minutes, or less than 10 minutes, or less than 20 minutes, or less than 30 minutes, or less than 40 minutes, or less than 50 minutes, or less than one hour, or less than 2 hours, or less than 3 hours) periods of time when the reported glucose values inaccurately reflect actual glucose concentration sensed by the CGM sensor.

Thus, discussed herein, accelerometer data may be relied upon for determination of user posture, which may be correlated with particular CGM sensor signal anomalies. As one example, particular postures as sensed by an accelerometer located at the position of the CGM sensor (e.g., coupled to the transmitter board) may result in a pressure trough, the length and depth of which is readily observable in the current data being retrieved from the CGM sensor. As a representative example, in a situation where the user is wearing the sensor on the front part of the abdomen (refer to the position of the CGM sensor at FIG. 5) and bends over to perform a task, this may result in a pressure artifact (e.g., pressure trough). This pressure artifact may last for as long as the user is bent over in the particular position. Such a pressure artifact may cause the current to deflect by up to 20% or even higher in some cases (e.g., 30% or more). Such an impact on current may result in reported glucose values changing by anywhere from 10-60 mg/dL, which of course is an undesirable situation and could trigger an alarm to alert the user of, for example, a hypoglycemic event. Because in reality the actual glucose concentration has not dropped, this may lead to the user taking undesirable action to compensate for this perceived drop in glucose levels. In other examples, changes in reported glucose values that are not reflective of actual glucose concentration sensed by the CGM sensor may result in an insulin pump, for example, being undesirably activated (for example if a postural disturbance results in what appears to be a hyperglycemic event, but in fact interstitial glucose levels are not elevated).

Thus, it is desirable to be able to detect and interpret such occurrences of signal artifacts by way of accelerometer data, and then take appropriate action (e.g., either do not display glucose values because they are not reliable, or correct/compensate the values as a function of the anomaly, so the reported glucose values accurately reflect the concentration of glucose sensed by the CGM sensor). In many instances, such postural disturbances that in turn cause CGM signal artifacts may be brief, as discussed above, for example 10 minutes or less or 5 minutes or less. A model (e.g., algorithm) may be employed to adaptively track such circumstances, and in turn, report corrected/compensated glucose values that accurately reflect the concentration of glucose sensed by the CGM sensor.

It may be understood that the above-mentioned ability to rely on accelerometer data to correct for postural disturbances to CGM signal is due to the location of the accelerometer (e.g., positioned on the transmitter board located in a housing that sits atop the user's skin, underneath which the CGM sensor is implanted into the skin) with respect to the CGM sensor. For example, if the accelerometer were to be positioned differently, for example at the wrist of the user (e.g., included as part of a watch), or included in as part of a computing device (e.g., carried by the user), it may not be possible (or may be substantially more difficult is possible at all) to correlate accelerometer data with particular postures and in turn, correct for CGM signal disturbances based on particular postures as determined based on accelerometer data.

To rely on such accelerometer data, a CGM system of the present disclosure may correlate changes in CGM-based current with adjunct data provided by the accelerometer, to assign particular periods where corrective action may be taken to adaptively report corrected/compensated glucose values instead of reporting glucose values that are artifacts due to postural disturbances to CGM sensor function.

While the above-description in terms of postural disturbances to CGM current signals relied on accelerometer data, it is within the scope of this disclosure that additionally or alternatively to accelerometer data, one or more pressure sensor(s) may be used to obtain similar information. As one example, one or more pressure sensors may be mounted on the adhesive patches on the bottom of a body-worn unit (e.g., a bottom of a housing that houses the transmitter board). In such an example, one or more temperature sensors may additionally or alternatively be attached to the adhesive patch.

Accordingly, at block 725, method 700 includes processing the accelerometer data retrieved from the accelerometer. This may be done by way of a model that accounts for predetermined patterns of accelerometer data in conjunction with predetermined patterns of current signal provided via the CGM sensor. The processing may thus involve assigning a particular time period to an event comprising a postural disturbance that impacts CGM-based signal current, and adaptively correcting/compensating reported glucose values so that the reported glucose values more accurately reflect the actual concentration of glucose sensed by the CGM sensor.

Thus, block 730 includes adaptively correcting/compensating reported glucose values based on the retrieved temperature data and/or accelerometer data, in conjunction with the retrieved CGM current data. In some examples, the adaptive compensation may take into account more than one type of data, for example temperature sensor data and accelerometer data, as there may be circumstances where accounting for both accelerometer and temperature data may further improve accuracy of reported glucose values in terms of actual glucose concentrations sensed by the CGM sensor. In examples, adaptively compensating glucose values at block 730 relies on the use of one or more correction factors derived from the data acquired and processed prior to block 730.

At block 735, method 700 includes storing relevant data. For example, it may be understood that data collected from the temperature sensor(s) and the accelerometer (and/or pressure sensor(s)) in combination with CGM current may be useful for refining the models used in method 700, and may additionally or alternatively be useful for aspects of CGM system operation. For example, data collected may comprise historical data useful for the methodology of FIG. 3, in some examples.

At block 740, method 700 includes controlling one or more actuators based on corrected/compensated glucose values. For example, similar to that discussed above, corrected glucose values may be used to prevent an alarm from being activated (e.g., auditory and/or vibratory) that would otherwise indicate a hyperglycemic or hypoglycemic event. For example, provided corrected glucose values are maintained within predetermined thresholds, an alarm may be prevented from being activated, that would otherwise have been activated if the reported glucose values were not compensated for. Similar logic applies to, for example, an insulin pump. Provided corrected glucose values do not exceed a hyperglycemic threshold, for example, then an insulin pump may not be activated, whereas it may otherwise be activated to deliver a bolus of insulin in the absence of the adaptive compensation methodology herein disclosed. Similar to that discussed above with regard to method 300 at FIG. 3, a user may be provided some indication that the values that are being reported comprise corrected/compensated values. Examples may include but are not limited to a color change in the reported values displayed on a visual display, flashing values as compared to non-flashing values, and the like. In some examples, some descriptive wording may be displayed in order to alert the user that the reported glucose values comprise corrected/compensated values. Furthermore, the reported values may be associated with a particular confidence level (e.g., high, medium, or low, etc.), such that the user may be apprised of how accurate the corrected/compensated values are likely to be. In some examples, one or more thresholds for controlling an actuator (e.g., insulin pump, alarm, etc.) may comprise adjustable thresholds, which may be adjusted to more conservative levels during time periods where the reported values comprise compensated values, and adjusted to less conservative levels during time periods when the values are not being adaptively compensated/corrected. In some examples, the degree to which the one or more thresholds are adjusted may be a function of the confidence level of the corrected/compensated reported glucose values. For example, the higher the confidence, the lesser a threshold may be adjusted.

At block 745, method 700 determines whether the CGM sensor has been removed, or if there is some other confounding issue detected (e.g., sensor degradation). If so, method 700 may end, and then may be restarted once the sensor has been replaced. Alternatively, if the CGM sensor has not been removed and no confounding issues are detected, method 700 returns to step 705 where the process flow of FIG. 7 repeats to adaptively correct for temperature and/or postural artifacts in relation to CGM sensor signals.

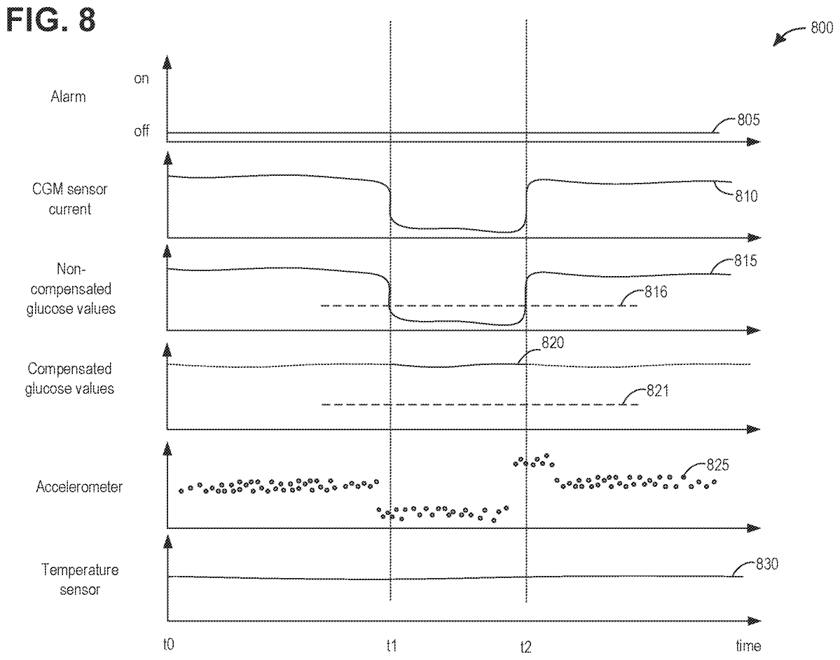

FIG. 8 depicts an example timeline 800 illustrating how accelerometer data may be used with a CGM system of the present disclosure, to detect and compensate for CGM sensor signal artifacts that are induced as a result of a particular user posture or activity a user is engaged in. In this example timeline, the accelerometer data is used in conjunction with at least CGM sensor current data, to compensate/correct reported glucose values, and in turn control an alarm that is used to alert a user to a particular condition (e.g., hyperglycemic or hypoglycemic event) based on the compensated/corrected glucose values.

Example timeline 800 includes plot 805, indicating whether the alarm (e.g., audible and/or vibrational alarm) is activated (on) or deactivated (off), over time. Timeline 800 further includes plot 810, indicating CGM sensor current, over time. Timeline 800 further includes plot 815, indicating non-compensated glucose values, and plot 820, indicating compensated glucose values, over time. Timeline 800 further includes plot 825, indicating accelerometer data retrieved from an accelerometer positioned at the transmitter board associated with a computing device (e.g., computing device 110 at FIG. 1) of a body-worn CGM device, over time. Timeline 800 further includes plot 830, indicating data retrieved from a temperature sensor of the CGM system, over time. In this example timeline, data from just one temperature sensor is indicated for clarity, and the temperature sensor may be understood to comprise a temperature sensor configured to monitor temperature of electronics associated with the computing device (e.g., temperature sensor positioned at the transmitter board). In this example timeline, plot 820 includes two sections that are illustrated by dotted lines, and one section that is illustrated by a solid line. This is to indicate that compensated glucose values may be substantially the same as non-compensated glucose values during times when no CGM sensor signal artifact is detected, but differ from non-compensated glucose values during times when a CGM sensor signal artifact is detected. In this example timeline, the CGM sensor signal artifact is recognized via a combination of at least CGM sensor current and accelerometer data, and may take into account the data retrieved from the temperature sensor in arriving at the determination of the signal artifact. Thus, the signal artifact described with regard to FIG. 8 may be understood to be an artifact that is due to a particular posture of the user.

Between time t0 and t1, the alarm is off, and accelerometer data is relatively stable (plot 825). CGM sensor current (plot 810) is also relatively stable, reflective of unchanging interstitial glucose concentration, and hence, non-compensated glucose values (plot 815) accurately represent glucose concentrations sensed by the CGM sensor.

At time t1, CGM sensor current (plot 810) is artificially impacted by the user adopting a posture that is reflected in the accelerometer data (plot 825). The pattern of current drop and accelerometer data, and absence of temperature sensor changes as indicated by plot 830, is interpreted as a predetermined postural effect and is characterized as such. Accordingly, the methodology of FIG. 7 is used to adaptively correct/compensate the reported glucose values (refer to plot 820) during the time period (time spanning t1 and t2) that the user is adopting the particular posture that is causing the artifact in CGM sensor current. Depicted at timeline 800 is line 816, representing a hypoglycemic threshold, below which the alarm is activated. Line 821 represents the same threshold, but is reproduced for clarity. Because the compensated glucose values remain above the threshold (line 821), the alarm is not activated, but otherwise would have been (refer to plot 815 with respect to line 816) if the reported glucose values were not compensated for based on at least the CGM sensor current and accelerometer data. After time t2, it is determined that the event that resulted in the posture-induced signal artifact is no longer present, and the glucose values reported once again comprise non-compensated values.

In some examples, a CGM system of the present disclosure may continually generate both compensated and non-compensated values, and divergence past a predetermined amount (e.g., values differ by more than 2%, or by more than 5%, or by more than 10%, etc.) may cause the system to rely on the compensated values as opposed to the non-compensated values.

The above description has laid out how adjunctive data may be used to improve quality and accuracy of CGM systems of the present disclosure, by correcting/compensating reported glucose values under circumstances where reported glucose values may otherwise not accurately reflect the actual concentration of glucose sensed by the CGM sensor. Yet it is herein recognized that there may be still further uses of adjunct data as herein disclosed. Specifically, the adjunct data may be useful in terms of projecting glucose values at a future time. This sort of data may comprise analysis of historical data trends as described in detail above, such that the data can be mined to predict particular combinations of data patterns retrieved from the one or more adjunct sensor(s) and CGM sensor current (or voltage) with regard to projected future glucose values. Adjunct data in the context of such future projected glucose values may comprise any or all of the types of adjunctive data herein disclosed (e.g., temperature sensor(s), pressure sensor(s), accelerometer(s), heart rate sensor, blood pressure sensor, data retrieved from software applications such as geographical location data and the like, etc.). Learning strategies (e.g., AI-based such as the above-referenced categories of machine-learning) based on historical data trends may be used to infer specific data patterns that are predictive of future glucose values with some associated level of confidence in the prediction. This may be particularly useful in terms of controlling alarms/alerts.

For example, based on particular recognized patterns of data retrieved from one or more adjunct sensors and CGM current data (e.g., CGM raw data stream), a CGM system of the present disclosure may be capable of predicting when a user may be going into a hypo or hyperglycemic episode. This type of projection may be advantageous to users in that the user may be alerted as to such an upcoming episode, so that they may take mitigating action prior to the event occurring. For example, meals or snacks may take a certain amount of time to have the full effect on blood glucose levels, thus an ability to know approximately (e.g., within 5 minutes or less, or within 10 minutes or less) when a hypo or hyperglycemic event may be expected to be occurring may enable a user to take more appropriate mitigating action as opposed to a situation where the user is not aware of such an upcoming event. As an example, user activity data derived from the accelerometer data may enable a CGM system of the present disclosure to infer a level of activity (e.g., high intensity workout), that may indicate a significant upcoming change in glucose levels at a predicted/inferred time in the future. This may be particularly relevant to those individuals afflicted with diabetes (e.g., Type I or Type II). Thus, combining such data with a predictive algorithm may provide much improved glucose prediction for users of CGM systems of the present disclosure. For example, this type of predictive modeling may be much more reliable than simply depending on a rate of change of glucose sensed by a CGM sensor, as such a rate of change measurement may trail the particular activity, and in combination with the lag time (time for plasma glucose to be fully reflected in an approximately equivalent change in interstitial fluid glucose) may result in predictions of future glucose values that may not be accurate or useful to users of CGM systems.

It is furthermore herein recognized that adjunctive data as herein disclosed may be used to improve quality of data with regard to the manner in which noise in the system is reduced. One example of how noise in a CGM system can be reduced is by way of averaging methodologies. For example, averaging over longer time periods can in some instances be helpful for noise reduction, however longer averaging times can undesirably contribute to introduction of additional lag time in the reported CGM data as compared to levels of blood glucose. It is herein recognized that via the reliance on adjunctive data in combination with CGM raw data streams, different noise filtering methodologies may be tailored to use during particular use-based circumstances. One example of a use-based circumstance may be a very high level of activity (e.g., high intensity workout), as identified by the accelerometer data. In an embodiment, upon detection of a particular pattern of activity such as a high intensity workout, the CGM system may shift to reliance on a noise-reduction technique (e.g., data filtering technique) that is appropriate for periods of high activity and potentially high levels of glucose change. Then, when activity level has subsided and/or rates of change in glucose level are lessened, the system may once again shift to reliance on a different noise-reduction technique that is more appropriate for periods of lower activity and/or lower levels of blood glucose changes.

### EXAMPLES

### Example 1

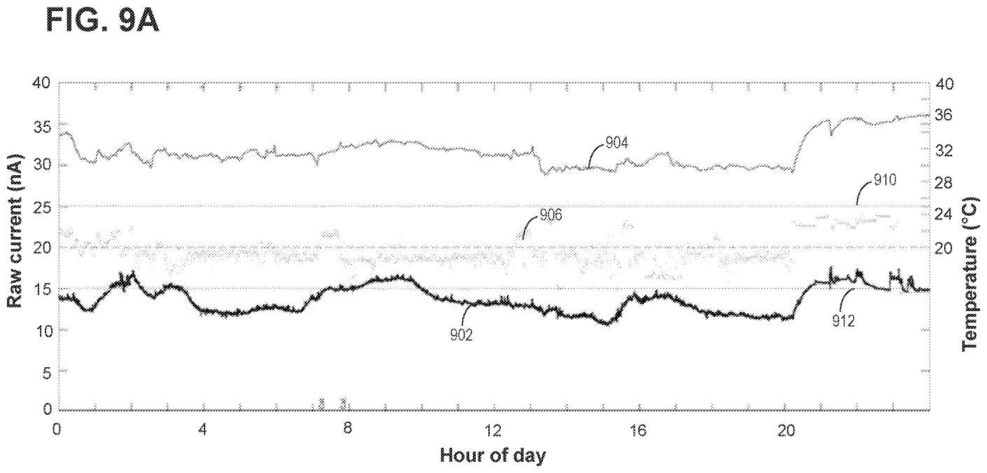

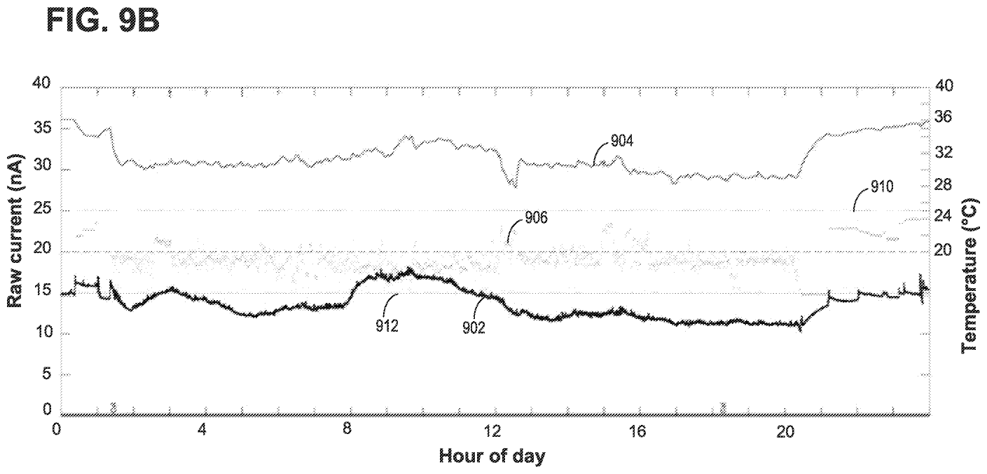

This Example demonstrates a correlation between data acquired via an analyte sensor, an accelerometer, and a temperature sensor. Depicted at FIG. 9A and FIG. 9B is current 902 corresponding to raw data obtained from the analyte sensor, a temperature trace 904 corresponding to data obtained via the temperature sensor, and movement data 906 obtained from the accelerometer. With regard to the accelerometer data, upper bound 910 and lower bound 912 are shown, comprising operationally defined bounds. The x-axis of each of FIG. 9A and FIG. 9B refers to hours of the day, and the y-axis refers to raw current in nA (left y-axis) and temperature in ° C. (right y-axis). FIG. 9A illustrates a 24 hour period corresponding to day 5 post analyte sensor insertion, and FIG. 9B illustrates a 24 hour period corresponding to day 6 post analyte sensor insertion.

At FIG. 9A, just after hour 20, there is a sharp rise in temperature that corresponds to a concomitant rise in current from the analyte sensor. The rise in both current and temperature plateau at about 21-22 hours. Turning to FIG. 9B, the pattern of temperature and current being correlated continues until about hour 1.5-2 of the following day (day 6). Again, at about hour 20 of day 6, a similar rise in temperature and concomitant rise in current associated with the analyte sensor is observed.

When correlated with the accelerometer data, it is clear that the time period during the concomitant rise in temperature and analyte sensor current corresponded to a period of very little activity. The user confirmed that this time period corresponded to a period of sleeping in which the user was covered by blankets. This in turn resulted in the temperature rise, and hence the rise in analyte sensor-derived current. If not corrected for, this may lead to a reporting of inaccurate glucose values, and other undesirable issues such as the triggering of an alarm and/or insulin pump operation. However, via the use of methodology herein disclosed, such a pattern of activity may be learned to comprise a situation where the current rise does not reflect an actual rise in glucose, such that mitigating action may be taken to avoid undesirable actions being taken such as activation of an insulin pump, triggering of an alarm, and the like.

In this way, a CAM system (e.g., CGM system) may operate with corrected analyte values during times when it is predicted that non-corrected analyte values may not be reflective of actual analyte concentration sensed by the particular continuous analyte sensor. The technical effect of predicting times when determined analyte values are expected to be inaccurate is that a number of adverse situations which otherwise may result if such a strategy is not employed, may be avoided. For example, by employing the methodology disclosed herein, users of CAM systems may avoid taking unnecessary action to manage analyte levels at times when such action is not actually called for. This may improve the safety profile associated with CAM systems as herein disclosed, and hence increase user satisfaction. The methodology may further improve user satisfaction by avoiding situations where an alarm, for example, disturbs the user unnecessarily. This is particularly relevant during times of sleeping, or driving (as examples), where the disturbance (if not representative of the underlying biology) may have adverse implications on the health and/or safety of the user.

Although various example methods, apparatus, systems, and articles of manufacture have been described herein, the scope of coverage of the present disclosure is not limited thereto. On the contrary, the present disclosure covers all methods, apparatus, and articles of manufacture fairly falling within the scope of the appended claims either literally or under the doctrine of equivalents. For example, although the above discloses example systems including, among other components, software or firmware executed on hardware, it should be noted that such systems are merely illustrative and should not be considered as limiting. In particular, it is contemplated that any or all of the disclosed hardware, software, and/or firmware components can be embodied exclusively in hardware, exclusively in software, exclusively in firmware or in some combination of hardware, software, and/or firmware.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method, comprising:
obtaining a first data stream corresponding to a concentration of an analyte in a biological fluid from an analyte sensor;
converting the first data stream into analyte values reflective of the concentration of the analyte;
obtaining one or more additional data streams from one or more adjunctive sensors;
inferring, based on the first data stream and the one or more additional data streams, that conversion of the first data stream into analyte values is predicted to be inaccurate; and
taking mitigating action to avoid inaccurate analyte values from being reported to a user.

2. The method of claim 1, wherein the one or more adjunctive sensors are selected from a pressure sensor, a temperature sensor, an accelerometer, and a heart rate sensor.

3. The method of claim 1, wherein inferring that conversion of the first data stream into analyte values is predicted to be inaccurate further comprises:
comparing the first data stream and the one or more additional data streams to a set of historical data that has been computationally processed to reveal patterns of data corresponding to analyte and adjunctive sensor data streams indicative of circumstances where conversion of acquired data into analyte values is inaccurate.

4. The method of claim 3, wherein computationally processing the set of historical data further comprises performing computational operations selected from one or more of supervised learning, unsupervised learning, and reinforcement learning, on the set of historical data.

5. The method of claim 1, wherein taking mitigating action further comprises:
applying a correction factor to a function that converts the first data stream into analyte values; and
reporting corrected analyte values to the user.

6. The method of claim 5, wherein reporting corrected analyte values to the user further comprises:
providing to the user an indication of a confidence level of the corrected analyte values.

7. The method of claim 5, further comprising:
preventing an alarm associated with the analyte sensor from being activated when the corrected analyte values do not exceed one or more predetermined analyte value thresholds.

8. The method of claim 1, wherein taking mitigating action further comprises:
alerting the user that the analyte values are currently inaccurate; and
providing a request to the user to obtain analyte values via another method that does not involve the analyte sensor.

9. The method of claim 1, wherein the analyte sensor is a continuous analyte sensor implanted interstitially in skin of the user.

10. The method of claim 1, wherein the analyte is glucose.

11. A method of controlling an actuator associated with a continuous glucose sensor system comprising:
predicting that conversion of a raw data stream obtained from a continuous glucose sensor interstitially implanted into skin of a user is expected to result in reporting of inaccurate glucose values that are not representative of an actual concentration of glucose sensed by the continuous glucose sensor;
applying a correction factor to a function that converts the raw data stream into glucose values, to obtain corrected glucose values that more accurately reflect the actual concentration of glucose sensed by the continuous glucose sensor, within a predetermined threshold range of the actual concentration;
controlling the actuator in a first mode when the corrected glucose values do not exceed one or more predetermined glucose value thresholds; and
controlling the actuator in a second mode when the corrected glucose values exceed at least one of the predetermined glucose value thresholds.

12. The method of claim 11, wherein the actuator is an alarm that is audible and/or vibrational; and
wherein controlling the alarm in the first mode includes preventing the alarm from being activated, and wherein controlling the alarm in the second mode includes activating the alarm to alert the user of a hypoglycemic or hyperglycemic event.

13. The method of claim 11, wherein the actuator is an insulin pump operably coupled to the continuous glucose sensor system, and capable of delivering a variable amount of insulin to the user as a function of determined glucose values; and
wherein controlling the insulin pump in the first mode includes maintaining the insulin pump off, and wherein controlling the insulin pump in the second mode includes activating the insulin pump as a function of an extent to which the corrected glucose values exceeds one of the predetermined glucose value thresholds corresponding to a hyperglycemic event.

14. The method of claim 11, wherein the predicting is based at least in part on data currently being acquired from the continuous glucose sensor and from at least one adjunct sensor, and a correlation of the data currently being acquired from both the continuous glucose sensor and the at least one adjunct sensor with previously obtained data that includes data obtained from the at least one adjunct sensor and the continuous glucose sensor or other similar adjunct sensor(s) and continuous glucose sensor(s) used in previous sensor sessions.

15. The method of claim 14, wherein the one or more adjunct sensors include a pressure sensor, a temperature sensor, and an accelerometer; and
wherein each of the one or more adjunct sensors and the continuous glucose sensor are all positioned on the user within a same area defined by a radius R, where radius R is 2 cm or less.

16. The method of claim 14, further comprising processing the previously obtained data via a computational strategy capable of learning when particular continuous glucose sensor data trends in combination with particular adjunct sensor data trends lead to inaccurate glucose values in absence of the correction factor.

17. The method of claim 11, further comprising providing a confidence level reflective of the corrected glucose values.

18. The method of claim 17, further comprising adjusting the one or more predetermined glucose value thresholds as a function of the confidence level of the corrected glucose values.

19. A glucose sensor system comprising:
a continuous glucose sensor for interstitial implantation into skin of a user;
one or more adjunct sensors selected from a pressure sensor, a temperature sensor, an accelerometer, and a heart rate sensor;
one or more actuatable components; and
a computing device storing instructions in non-transitory memory that, when executed, cause the computing device to:
retrieve a first data stream from the continuous glucose sensor;
retrieve one or more additional data streams from the one or more adjunct sensors;
compare the first data stream and the one or more additional data streams to a historical data set comprising learned associative patterns of data corresponding to previously acquired data from the continuous glucose sensor and the one or more adjunct sensors, wherein the learned associative patterns are related to instances where conversion of the first data stream into glucose values results in glucose values that are not reflective of actual glucose concentrations measured via the continuous glucose sensor;
predict, based on the comparing, that converting the first data stream into glucose values is expected to result in glucose values that are not reflective of the actual glucose concentrations measured via the continuous glucose sensor;
initiate a compensation operation to yield corrected glucose values that are reflective of the actual glucose concentrations within some threshold of the actual glucose concentrations; and
control at least one of the one or more actuatable components based on the corrected glucose values under circumstances where the compensation operation can yield the corrected glucose values that are reflective of the actual glucose concentrations within the threshold of the actual glucose concentrations.

20. The system of claim 19, further comprising:
a display operably linked to the computing device; and
wherein the computing device stores further instructions to send the corrected glucose values to the display device for viewing by the user, along with an indication that the values correspond to the corrected glucose values.

21. The system of claim 20, wherein the indication that the values correspond to the corrected glucose values includes one or more of displaying the corrected glucose values in a flashing manner as opposed to a stable manner, displaying the corrected glucose values in a color that is different from when non-corrected glucose values are displayed, and displaying a message along with the corrected glucose values that provides the user with information indicating that values displayed correspond to the corrected glucose values.

22. The system of claim 19, wherein the computing device stores further instructions to:
prevent a calibration operation from being initiated during a time frame when the first data stream is being converted via the compensation operation to the corrected glucose values; and
reschedule the calibration operation for another time under conditions where the calibration operation was scheduled to occur during the time frame when the first data stream is being converted to the corrected glucose values.

23. The system of claim 19, wherein the computing device stores further instructions to:
assign a confidence level to the corrected glucose values; and
control at least one of the one or more actuatable components based in part on the confidence level assigned to the corrected glucose values.

24. The system of claim 19, wherein the actuatable component is an audible and/or vibrational alarm configured to alert the user of a biological event related to blood glucose levels;
wherein the computing device stores further instructions to prevent the alarm from being activated provided that the corrected glucose values do not exceed one or more predetermined glucose value thresholds; and
activate the alarm in response to the corrected glucose values exceeding the one or more predetermined glucose value thresholds for a predetermined amount of time.

25. The system of claim 19, wherein the actuatable component is an insulin pump operably linked to the computing device; and
wherein the computing device stores further instructions to prevent the insulin pump from being activated provided that the corrected glucose values do not exceed a hyperglycemic threshold; and
activate the insulin pump according to stored instructions in response to the corrected glucose values exceeding the hyperglycemic threshold for a predetermined amount of time.

26. The system of claim 19, wherein the computing device stores further instructions to:
compare the first data stream and the one or more additional data streams to the historical data set, the historical data set additionally comprising learned associative patterns of data related to instances where conversion of the first data stream into glucose values results in glucose values that are accurately reflective of actual glucose concentrations measured via the continuous glucose sensor; and
control at least one of the one or more actuatable components based on non-corrected glucose values under circumstances where it is predicted that the non-corrected glucose values are reflective of actual glucose concentrations.

27. A method for a continuous analyte sensor system, comprising:
determining, based on a first data stream retrieved from a continuous analyte sensor and at least a second data stream retrieved from an adjunct sensor, that a user of the continuous analyte sensor system has adopted a posture that results in the first data stream inaccurately reflecting a concentration of an analyte sensed by the continuous analyte sensor;
providing, based on at least the first data stream and the second data stream, compensated analyte values that accurately reflect the concentration of the analyte sensed by the continuous analyte sensor during a time period that the user is adopting the posture; and
controlling at least one actuator of the continuous analyte sensor system based on the compensated analyte values during the time period that the user is adopting the posture.

28. The method of claim 27, wherein the adjunct sensor is an accelerometer.

29. The method of claim 28, wherein the accelerometer is comprised of a chip that is attached to a transmitter board circuit included in a housing that is worn on the skin of the user, and which sits atop a location where the continuous analyte sensor is inserted into the skin of the user.

30. The method of claim 27, wherein the adjunct sensor comprises one or more pressure sensors.

31. The method of claim 30, wherein the one or more pressure sensors are coupled to an adhesive patch used to secure a housing to the skin of the user, and which sits atop a location where the continuous analyte sensor is inserted into the skin of the user.

32. The method of claim 27, further comprising detecting, based at least on the first data stream and the second data stream, that the user is no longer adopting the posture; and
providing non-compensated analyte values that accurately reflect the concentration of the analyte sensed by the continuous analyte sensor.

33. The method of claim 27, wherein the at least one actuator comprises an alarm configured to alert the user of an adverse event related to blood levels of the analyte.

34. The method of claim 33, further comprising preventing the alarm from notifying the user of the adverse event provided that the compensated analyte values do not exceed one or more predetermined analyte value thresholds.

35. The method of claim 27, wherein the analyte is glucose; and
wherein the continuous analyte sensor system is a continuous glucose monitoring system.

36. The method of claim 27, further comprising retrieving data from the adjunct sensor at intervals of between 10-20 seconds.

37. A method for a continuous analyte sensor system, comprising:
retrieving a first data stream corresponding to current that is reflective of a concentration of an analyte sensed by a continuous analyte sensor;
converting the first data stream into analyte values reflective of the concentration of the analyte sensed by the continuous analyte sensor;
retrieving one or more additional data streams from one or more additional temperature sensors positioned within a predetermined distance of the continuous analyte sensor;
determining, based on the one or more additional data streams, that conversion of the first data stream is predicted to result in analyte values that do not accurately reflect the concentration of the analyte sensed by the continuous analyte sensor; and
providing compensated analyte values based on the one or more additional data streams that more accurately reflect the concentration of the analyte within a predetermined threshold range of the concentration of the analyte sensed by the continuous analyte sensor.

38. The method of claim 37, wherein the one or more additional data streams comprise a second data stream retrieved from a first temperature sensor positioned on a transmitter board contained within a housing that is part of the continuous analyte sensor system, the housing configured to be attached to skin of the user and sit atop the continuous analyte sensor when the continuous analyte sensor is inserted into the skin of the user; and
wherein providing the compensated analyte values comprises utilizing a characterized temperature sensitivity of one or more temperature-sensitive electronic components that can adversely impact the first data stream, and temperature values corresponding to the second data stream, in a model that in turn outputs the compensated analyte values.

39. The method of claim 37, wherein the one or more additional data streams comprise a third data stream retrieved from a second temperature sensor positioned on a surface of the skin within the predetermined distance of the continuous analyte sensor; and
wherein providing the compensated analyte values comprises incorporating into a model that outputs the compensated analyte values a user-specific lag time corresponding to a time delay between when plasma analyte values are reflected in an equivalent change in interstitial fluid analyte levels, the user-specific lag time a function of temperature values corresponding to the third data stream.

40. The method of claim 37, wherein the one or more additional data streams comprise a fourth data stream retrieved from a third temperature sensor positioned on a portion of the continuous analyte sensor that is inserted into the skin of the user; and
wherein providing the compensated analyte values comprises relying on the fourth data stream to infer a diffusion rate of the analyte into the sensor, and incorporating the inferred diffusion rate into a model that outputs the compensated analyte values.

41. The method of claim 37, wherein the analyte is glucose; and
wherein the continuous analyte system is a continuous glucose monitoring system.

42. The method of claim 37, wherein providing the compensated analyte values is based at least in part on the current corresponding to the first data stream.

43. The method of claim 37, wherein the predetermined distance is 2 cm or less.

44. A method for a continuous analyte sensor system, comprising:
retrieving a first data stream from a continuous analyte sensor configured to sense an analyte concentration in an interstitial fluid of a user;
retrieving one or more additional data streams from one or more adjunct sensors positioned within a predetermined distance from the continuous analyte sensor;
comparing the first data stream and the one or more additional data streams to a set of historical data that has been computationally processed to reveal patterns of data corresponding to the first and the one or more additional data streams indicative of a future event related to blood analyte levels; and
providing an alert to the user that the future event is predicted to occur within a determined time frame.

45. The method of claim 44, wherein the analyte is glucose; and
wherein the continuous analyte system is a continuous glucose monitoring system.

46. The method of claim 45, wherein the future event is one of a hypoglycemic event or a hyperglycemic event.

47. The method of claim 44, wherein the determined time frame is between 30 minutes to 90 minutes.

48. The method of claim 44, wherein the one or more adjunct sensors are selected from an accelerometer, one or more temperature sensors, one or more pressure sensors, a hear rate sensor, and a blood pressure sensor.
